SEQUENCE LISTING (12 sequences)

SEQ ID NO: 1 - protein, 5 aa, human
NSGMN

SEQ ID NO: 2 - protein, 17 aa, human
WINTYNGEPTYTDDFKG

SEQ ID NO: 3 - protein, 11 aa, human
HSYGSSYAMDN

SEQ ID NO: 4 - protein, 15 aa, human
KASQSVDYDGDSYMN

SEQ ID NO: 5 - protein, 7 aa, human
VASNLKS

SEQ ID NO: 6 - protein, 9 aa, human
QQSNEEPWT

SEQ ID NO: 7 - protein, 120 aa, human
QIQLVQSGPELKKPGASVKVSCRASGYPFTNSGMNWVKQAPGKGLKWMGWINTYNGEPTYTDDFKGRFAFSLETSASTAYLQINNLRNEDTATYFCASHSYGSSYAMDNWGQGTSVTVSS

SEQ ID NO: 8 - protein, 111 aa, human
DIVLTQSPDSLAVSLGERATINCKASQSVDYDGDSYMNWYQQKPGQPPKLLIYVASNLKSGIPARFSGSGSGTDFTLTISSLQPEDFATYYCQQSNEEPWTFGGGTKVEIK

SEQ ID NO: 9 - DNA, 360 bp, human
cagattcagctggtgcagtccggccctgagctgaagaaacctggcgcctccgtgaaggtgtcctgcagagcttccggctaccccttcaccaactccggcatgaactgggtcaagcaggcccctggcaagggcctgaagtggatgggctggatcaacacctacaacggcgagcccacctacaccgacgacttcaagggcagattcgccttctccctggaaacctccgcctccaccgcctacctgcagatcaacaacctgcggaacgaggacaccgccacctacttttgcgcctcccactcctacggctccagctacgccatggacaattggggccagggcacctccgtgaccgtgtctagc

SEQ ID NO: 10 - DNA, 333 bp, human
gatatcgtgctgacccagtcccctgactccctggctgtgtctctgggcgagagagccaccatcaactgcaaggcctcccagtccgtggactacgacggcgactcctacatgaactggtatcagcagaagcccggccagccccccaagctgctgatctacgtggcctccaacctgaagtccggcatccctgccagattctccggctctggctctggcaccgactttaccctgaccatctccagcctgcagcccgaggacttcgccacctactactgccagcagtccaacgaggaaccctggacctttggcggaggcaccaaggtggaaatcaag

SEQ ID NO: 11 - protein, 450 aa, human
QIQLVQSGPELKKPGASVKVSCRASGYPFTNSGMNWVKQAPGKGLKWMGWINTYNGEPTYTDDFKGRFAFSLETSASTAYLQINNLRNEDTATYFCASHSYGSSYAMDNWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, human
DIVLTQSPDSLAVSLGERATINCKASQSVDYDGDSYMNWYQQKPGQPPKLLIYVASNLKSGIPARFSGSGSGTDFTLTISSLQPEDFATYYCQQSNEEPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0235037 A1
(43) Pub. Date: Jul. 27, 2023

(54) METHOD OF TREATMENT USING ANTI-CCL24 ANTIBODY

(71) Applicant: CHEMOMAB LTD., Tel Aviv (IL)

(72) Inventors: Adi MOR, Tel Aviv (IL); Arnon AHARON, Tel Aviv (IL); Sharon HASHMUELI, Ramot Naftaly (IL); Michal SEGAL SALTO, Tel Aviv (IL); Neta BARASHI, Tel Aviv (IL)

(73) Assignee: CHEMOMAB LTD., Tel Aviv (IL)

(21) Appl. No.: 17/996,386

(22) PCT Filed: Apr. 20, 2021
(86) PCT No.: PCT/IL2021/050447

§ 371 Date: Oct. 17, 2022

Related U.S. Application Data
(60) Provisional application No. 63/013,793, filed on Apr. 22, 2020.

(51) Int. Cl. C07K 16/24; A61K 47/26; A61K 47/18; A61K 9/00; A61P 1/16

(52) U.S. Cl. CPC C07K 16/24; A61K 47/26; A61K 47/183; A61K 9/0019; A61P 1/16

(57) ABSTRACT

Provided are compositions and methods for treating fibrotic inflammatory diseases, including non-alcoholic steatohepatitis (NASH), primary sclerosing cholangitis (PSC) and systemic sclerosis (SSc), with an anti-CCL24 monoclonal antibody.

### FIELD OF INVENTION

The disclosure relates generally to the use of an anti-CCL24 monoclonal antibody to treat fibrotic inflammatory diseases in humans.

### BACKGROUND

Non-alcoholic fatty liver disease (NAFLD) is the most common chronic liver disease in the world, affecting up to 30% of the adult population and 70-80% of individuals who are obese and diabetic (Chalasani et al. 2012). NAFLD encompasses a histological spectrum, ranging from simple steatosis to non-alcoholic steatohepatitis (NASH), the latter of which can have different degrees of fibrosis severity. Although many individuals with simple steatosis do not develop more severe disease, NASH can progress to both cirrhosis and end-stage liver disease. NASH is the second leading etiology of liver disease among adults awaiting liver transplantation in the United States and is projected to become the most common indication for liver transplantation by 2020 (Wong et al. 2015). Furthermore, NASH is an emerging risk factor for type-2 diabetes, cardiovascular disease and end-stage kidney disease (Chalasani et al. 2012, Musso et al. 2014).

Primary sclerosing cholangitis (PSC) is a rare, chronic cholestatic liver disease characterized by progressive inflammation, fibrosis, and destruction of the intrahepatic and extra-hepatic bile ducts with no identifiable cause. PSC disease progression can vary, but typically begins in portal tracts; fibrosis and commensurate inflammatory responses induce a progressive spread of the fibrotic condition. No treatment to date, aside from liver transplant, has been associated with an improvement in clinical outcome.

Systemic sclerosis (SSc) is a systemic connective tissue disease. Characteristics of systemic sclerosis include essential vasomotor disturbances; fibrosis; subsequent atrophy of the skin, subcutaneous tissue, muscles, and internal organs (eg, alimentary tract, lungs, heart, kidney, CNS); and immunologic disturbances. SSc is a complex rheumatologic autoimmune disease in which inflammation, fibrosis, and vasculopathy share several pathogenic pathways that lead to skin and internal organ damage.

CCL24 is a chemokine that promotes cell trafficking and regulates inflammatory and fibrotic activities through the CCR3 complex. CCL24 is produced by several types of cells and its receptor CCR3 is present on eosinophils, T-cells, monocytes and fibroblasts. CM-101 is a humanized monoclonal antibody against CCL24.

There remains a need in the art for improved methods of treating fibrotic inflammatory diseases such as non-alcoholic steatohepatitis (NASH), Primary sclerosing cholangitis (PSC) and Systemic sclerosis (SSc).

### SUMMARY

The present disclosure relates generally to dosage and timing of administration of an anti-CCL24 monoclonal antibody to treat fibrotic inflammatory diseases in human subjects.

In one aspect, the present disclosure provides methods of treating a fibrotic inflammatory disease in a human subject comprising administering an effective amount of an anti-CCL24 monoclonal antibody. The present disclosure also provides an anti-CCL24 monoclonal antibody for use in methods of treating a fibrotic inflammatory disease in a human subject, said method comprising administering an effective amount of said anti-CCL24 monoclonal antibody to the human subject. In some embodiments, anti-CCL24 monoclonal antibody comprises: a heavy chain variable region comprising: a) the complementary determining region VH CDR1 comprising the amino acid sequence denoted by SEQ ID NO: 1; b) the complementary determining region VH CDR2 comprising the amino acid sequence denoted by SEQ ID NO: 2; and c) the complementary determining region VH CDR3 comprising the amino acid sequence denoted by SEQ ID NO: 3; and a light chain variable region comprising: d) the complementary determining region VK CDR1 comprising the amino acid sequence denoted by SEQ ID NO: 4; e) the complementary determining region VK CDR2 comprising the amino acid sequence denoted by SEQ ID NO: 5; and f) the complementary determining region VK CDR3 comprising the amino acid sequence denoted by SEQ ID NO: 6. In some embodiments, the anti-CCL24 monoclonal antibody is a humanized or human antibody. In some embodiments, the anti-CCL24 monoclonal antibody comprises a heavy chain variable region having at least 95% sequence identity to SEQ ID NO:7. In some embodiments, the anti-CCL24 monoclonal antibody comprises a heavy chain having at least 95% sequence identity to SEQ ID NO: 11. In some embodiments, the anti-CCL24 monoclonal antibody comprises a light chain variable region having at least 95% sequence identity to SEQ ID NO: 8. In some embodiments, the anti-CCL24 monoclonal antibody comprises a light chain having at least 95% sequence identity to SEQ ID NO: 12. In some embodiments, the anti-CCL24 monoclonal antibody comprises a heavy chain variable region having 100% sequence identity to SEQ ID NO:7. In some embodiments, the anti-CCL24 monoclonal antibody comprises a heavy chain having 100% sequence identity to SEQ ID NO: 11. In some embodiments, the anti-CCL24 monoclonal antibody comprises a light chain variable region having 100% sequence identity to SEQ ID NO: 8. In some embodiments, the anti-CCL24 monoclonal antibody comprises a light chain having 100% sequence identity to SEQ ID NO: 12. In some embodiments, the anti-CCL24 monoclonal antibody comprises a heavy chain according to SEQ ID NO: 11 and a light chain according to SEQ ID NO: 12.

In some embodiments, the anti-CCL24 monoclonal antibody is administered at a dose of between about 0.75 mg/kg and about 20 mg/kg. In some embodiments, the anti-CCL24 monoclonal antibody is administered at a dose of about 10 mg/kg. In some embodiments, the anti-CCL24 monoclonal antibody is administered at a dose of about 10 mg/kg. In some embodiments, the anti-CCL24 monoclonal antibody is administered at a dose of about 5 mg/kg. In some embodiments, the anti-CCL24 monoclonal antibody is administered at a dose of about 2.5 mg/kg. In some embodiments, the anti-CCL24 monoclonal antibody is administered subcutaneously. In some embodiments, the anti-CCL24 monoclonal antibody is administered intravenously. In some embodiments, the anti-CCL24 monoclonal antibody is administered once every one to four weeks. In some embodiments, the anti-CCL24 monoclonal antibody is administered once every two weeks. In some embodiments, the anti-CCL24 monoclonal antibody is administered once every three weeks. In some embodiments, the anti-CCL24 monoclonal antibody is administered once every four weeks. In some embodiments, the subject maintains a steady-state serum concentration of the anti-CCL24 monoclonal antibody of about 10 to about 1000 μg/mL. In some embodiments, the subject maintains a steady-state serum concentration of the anti-CCL24 monoclonal antibody of about 60 to about 90 μg/mL. In some embodiments, the subject maintains a steady-state serum concentration of the anti-CCL24 monoclonal antibody of about 70 to about 300 μg/mL. In some embodiments, the anti-CCL24 monoclonal antibody reaches the steady-state serum concentration following three administrations.

In some embodiments, the anti-CCL24 monoclonal antibody is administered for at least four weeks. In some embodiments, the anti-CCL24 monoclonal antibody is administered for at least 15 weeks. In some embodiments, the anti-CCL24 monoclonal antibody is administered for at least 24 weeks. In some embodiments, the half-life of the anti-CCL24 monoclonal antibody is about 10-30 days. In some embodiments, the half-life of the anti-CCL24 monoclonal antibody is about 17 days. In some embodiments, the half-life of the anti-CCL24 monoclonal antibody is about 19 days. In some embodiments, the fibrotic inflammatory disease treated by the method of the disclosure is nonalcoholic steatohepatitis (NASH). In some embodiments, the fibrotic inflammatory disease treated by the method of the disclosure is primary sclerosing cholangitis (PSC). In some embodiments, the fibrotic inflammatory disease treated by the method of the disclosure is systemic sclerosis (SSC).

In some embodiments, administration of the anti-CCL24 monoclonal antibody increases total sera level of CCL24 at day 4 post administration to at least 150% of baseline total sera level of CCL24 before administering the anti-CCL24 monoclonal antibody. In some embodiments, administration of the anti-CCL24 monoclonal antibody improves hepatic condition of the subject as evaluated by one or more circulating fibrosis markers. In some embodiments, the one or more circulating fibrosis markers are selected from the group consisting of plasma alanine aminotransferase (ALT), alkaline phosphatase (ALP), aspartate aminotransferase (AST), PRO-C3, PRO-C4, PRO-05, C3M, cytokeratin 18 (CK18), amino-terminal propeptide of type III procollagen (PIIINP), hyaluronic acid (HA), Tissue Inhibitor of Metalloproteinases 1 (TIMP-1), Tissue Inhibitor of Metalloproteinases 2 (TIMP-2), platelet derived growth factor (PDGF) (e.g. PDGF AA comprising two alpha subunits), γ-glutamyltransferase (GGT), bilirubin, C-reactive protein (CRP), erythrocyte sedimentation rate (ESR), and procalcitonin (PCT). In some embodiments, the one or more circulating fibrosis markers are measured by Enhanced Liver Fibrosis (ELF) blood test. In some embodiments, administration of the anti-CCL24 monoclonal antibody decreases serum level of Tissue Inhibitor of Metalloproteinases 1 (TIMP-1) by at least 10% compared to baseline before administering the anti-CCL24 monoclonal antibody. In some embodiments, administration of the anti-CCL24 monoclonal antibody decreases serum level of Tissue Inhibitor of Metalloproteinases 2 (TIMP-2) by at least 5% compared to baseline before administering the anti-CCL24 monoclonal antibody. In some embodiments, administration of the anti-CCL24 monoclonal antibody decreases serum level of PDGF AA by at least 5% or at least 10% compared to baseline before administering the anti-CCL24 monoclonal antibody. In some embodiments, administration of the anti-CCL24 monoclonal antibody improves hepatic condition as evaluated by one or more imaging tests. In some embodiments, the one or more imaging tests comprise Magnetic Resonance Imaging Proton Density Fat Fraction (MRI-PDFF), liver elastography and/or FibroScan. In some embodiments, administration of the anti-CCL24 monoclonal antibody decreases liver fat content of the subject by at least 15% compared to baseline before administering the anti-CCL24 monoclonal antibody. In some embodiments, administration of the anti-CCL24 monoclonal antibody decreases liver stiffness by at least 0.5 kPa compared to baseline before administering the anti-CCL24 monoclonal antibody as measured by FibroScan. In some embodiments, administration of the anti-CCL24 monoclonal antibody improves hepatic condition as evaluated by one or more tests for liver enzyme function. In some embodiments, administration of the anti-CCL24 monoclonal antibody decreases γ-glutamyltransferase (GGT) level by at least 20% compared to baseline before administering the anti-CCL24 monoclonal antibody. In some embodiments, administration of the anti-CCL24 monoclonal antibody decreases alanine aminotransferase (ALT) level by at least 10% compared to baseline before administering the anti-CCL24 monoclonal antibody. In some embodiments, administration of the anti-CCL24 monoclonal antibody causes no dose limiting toxicity. In some embodiments, administration of the anti-CCL24 monoclonal antibody causes no injection site reaction. In some embodiments, administration of the anti-CCL24 monoclonal antibody causes no severe or serious adverse event (AE). In some embodiments, administration of the anti-CCL24 monoclonal antibody does not elevate level of triglycerides (TG), low-density lipoprotein (LDL) and/or Cholesterol in the subject's blood.

In one aspect, the present disclosure presents methods of treating nonalcoholic steatohepatitis (NASH) in a human subject comprising administering an effective amount of an anti-CCL24 monoclonal antibody or a pharmaceutical composition thereof. In some embodiments, the anti-CCL24 monoclonal antibody comprises a heavy chain according to SEQ ID NO: 11 and a light chain according to SEQ ID NO: 12. In some embodiments, the anti-CCL24 monoclonal antibody is administered at a dose of about 2.5 mg/kg, about 5 mg/kg or about 10 mg/kg. In some embodiments, the anti-CCL24 monoclonal antibody is administered at a dose of about 5 mg/kg. In some embodiments, the anti-CCL24 monoclonal antibody is administered subcutaneously. In some embodiments, the anti-CCL24 monoclonal antibody is administered once every two weeks. In some embodiments, the subject maintains a steady state serum concentration of the anti-CCL24 monoclonal antibody of about 60 to about 90 μg/mL. In some embodiments, the anti-CCL24 monoclonal antibody reaches the steady state serum concentration following 3 administrations. In some embodiments, the anti-CCL24 monoclonal antibody is administered for at least 14 weeks. In some embodiments, the half-life of the anti-CCL24 monoclonal antibody is about 16 days. In some embodiments, administration of the anti-CCL24 monoclonal antibody increases total sera level of CCL24 at day 4 post administration to at least 150% of baseline total sera level of CCL24 before administering the anti-CCL24 monoclonal antibody. In some embodiments, administration of the anti-CCL24 monoclonal antibody improves hepatic condition of the subject as evaluated by one or more circulating fibrosis markers. In some embodiments, administration of the anti-CCL24 monoclonal antibody decreases liver fat content of the subject compared to baseline before administering the anti-CCL24 monoclonal antibody as measured by MRI-PDFF. In some embodiments, administration of the anti-CCL24 monoclonal antibody decreases liver stiffness compared to baseline before administering the anti-CCL24 monoclonal antibody as measured by FibroScan.

In one aspect, the present disclosure provides methods treating primary sclerosing cholangitis (PSC) in a human subject comprising administering an effective amount of an anti-CCL24 monoclonal antibody or a pharmaceutical composition thereof. In some embodiments, the anti-CCL24 monoclonal antibody comprises a heavy chain according to SEQ ID NO: 11 and a light chain according to SEQ ID NO: 12. In some embodiments, the anti-CCL24 monoclonal antibody is administered at a dose of about 5 mg/kg, about 10 mg/kg, or about 20 mg/kg. In some embodiments, the anti-CCL24 monoclonal antibody is administered at a dose of about 10 mg/kg. In some embodiments, the anti-CCL24 monoclonal antibody is administered intravenously. In some embodiments, the anti-CCL24 monoclonal antibody is administered once every three weeks. In some embodiments, the subject maintains a steady state serum concentration of the anti-CCL24 monoclonal antibody of about 70 to about 300 μg/mL. In some embodiments, the anti-CCL24 monoclonal antibody reaches the steady state serum concentration following 3 administrations. In some embodiments, the anti-CCL24 monoclonal antibody is administered for at least 12 weeks. In some embodiments, the half-life of the anti-CCL24 monoclonal antibody is about 19 days. In some embodiments, administration of the anti-CCL24 monoclonal antibody lowers the level of steady state serum concentration of CCL24 compared to baseline before administering the anti-CCL24 monoclonal antibody. In some embodiments, administration of the anti-CCL24 monoclonal antibody improves hepatic condition of the subject as evaluated by one or more circulating fibrosis markers. In some embodiments, administration of the anti-CCL24 monoclonal antibody lowers the level of circulating alkaline phosphatase (ALP) compared to baseline before administering the anti-CCL24 monoclonal antibody. In some embodiments, administration of the anti-CCL24 monoclonal antibody lowers Enhanced Liver Fibrosis (ELF) score compared to baseline before administering the anti-CCL24 monoclonal antibody. In some embodiments, administration of the anti-CCL24 monoclonal antibody lowers the level of circulating PRO-C3, PRO-C4, C3M, and/or PRO-05 compared to baseline before administering the anti-CCL24 monoclonal antibody. In some embodiments, administration of the anti-CCL24 monoclonal antibody decreases liver stiffness compared to baseline before administering the anti-CCL24 monoclonal antibody as measured by FibroScan.

In one aspect, the present disclosure provides methods of treating systemic sclerosis (SSc) in a human subject comprising administering an effective amount of an anti-CCL24 monoclonal antibody or a pharmaceutical composition thereof. In some embodiments, the anti-CCL24 monoclonal antibody comprises a heavy chain according to SEQ ID NO: 11 and a light chain according to SEQ ID NO: 12. In some embodiments, the anti-CCL24 monoclonal antibody is administered at a dose of about 2.5-10 mg/kg. In some embodiments, the anti-CCL24 monoclonal antibody is administered intravenously. In some embodiments, the anti-CCL24 monoclonal antibody is administered once every two, three, or four weeks. In some embodiments, the subject maintains a steady state serum concentration of the anti-CCL24 monoclonal antibody of about 70 to about 300 μg/mL. In some embodiments, the anti-CCL24 monoclonal antibody reaches the steady state serum concentration following 3 administrations. In some embodiments, the anti-CCL24 monoclonal antibody is administered for at least 24 weeks. In some embodiments, administration of the anti-CCL24 monoclonal antibody increases total sera level of CCL24 at day 4 post administration to at least 150% of baseline total sera level of CCL24 before administering the anti-CCL24 monoclonal antibody. In some embodiments, administration of the anti-CCL24 monoclonal antibody improves Combined Response Index for Systemic Sclerosis (CRISS) score of the subject compared to baseline before administering the anti-CCL24 monoclonal antibody. In some embodiments, administration of the anti-CCL24 monoclonal antibody lowers the level of one or more circulating fibrosis markers compared to baseline before administering the anti-CCL24 monoclonal antibody. In some embodiments, administration of the anti-CCL24 monoclonal antibody improves lung function of the subject compared to baseline before administering the anti-CCL24 monoclonal antibody.

In one aspect, the present disclosure provides pharmaceutical compositions comprising about 1 to about 500 mg/mL of an anti-CCL24 monoclonal antibody comprising a heavy chain according to SEQ ID NO: 11 and a light chain according to SEQ ID NO: 12. In some embodiments, the pharmaceutical composition comprises about 100 to about 250 mg/mL the anti-CCL24 monoclonal antibody. In some embodiments, the pharmaceutical composition comprises about 175 mg/mL the anti-CCL24 monoclonal antibody. In some embodiments, the pharmaceutical composition comprises about 5 to about 20 mg/mL the anti-CCL24 monoclonal antibody. In some embodiments, the pharmaceutical composition comprises about 10 mg/mL the anti-CCL24 monoclonal antibody. In some embodiments, the pharmaceutical composition comprises a histidine buffer, optionally at about 10 mM. In some embodiments, the pharmaceutical composition comprises a sodium or potassium phosphate buffer, optionally at about 10 mM. In some embodiments, the pharmaceutical composition comprises arginine, optionally at between about 30 and about 200 mM, or optionally at about 150 mM. In some embodiments, the pharmaceutical composition comprises glutamic acid, optionally at between about 30 and about 200 mM, or optionally at about 150 mM. In some embodiments, the pharmaceutical composition comprises sorbitol, optionally at about 4%. In some embodiments, the pharmaceutical composition comprises glycine, optionally at about 10 mM to about 100 mM, or optionally about 40 mM. In some embodiments, the pharmaceutical composition comprises polysorbate, optionally polysorbate-80, optionally at about 0.02% or about 0.01%. In some embodiments, the pharmaceutical composition has a pH of 5 to 8. In some embodiments, the pharmaceutical composition has a pH of 6 to 7, optionally 6.1 or 6.3. In some embodiments, the pharmaceutical composition is supplied in a unit dose having a fill volume of about 0.2 to about 2 mL, or about 5 to about 20 mL.

In one aspect, the present disclosure provides methods of treating nonalcoholic steatohepatitis (NASH) in a human subject comprising administering an effective amount of the pharmaceutical composition of the present disclosure.

In one aspect, the present disclosure provides methods of treating primary sclerosing cholangitis (PSC) in a human subject comprising administering an effective amount of the pharmaceutical composition of the present disclosure.

In one aspect, the present disclosure provides methods of treating Systemic Sclerosis (SSc) in a human subject comprising administering an effective amount of the pharmaceutical composition of the present disclosure. In some embodiments, the administering step comprises subcutaneous administration. In some embodiments, the administering step comprises intravenous administration.

In one aspect, the present disclosure provides kits comprising the pharmaceutical composition of the disclosure and instructions for use.

### BRIEF DESCRIPTION OF DRAWINGS

FIG. 1 shows CM-101 average serum concentrations vs. time data, semi-log scale following a single IV administration

FIG. 2 shows CM-101 average serum concentrations vs. time data, semi-log scale. Following single 5 mg/kg IV or SC administration

FIG. 3 shows CM-101 average serum concentrations vs. time data, semi-log scale during 5 administrations of 2.5 mg/kg IV CM-101 once every 3 weeks

FIG. 4 depicts simulated and observed CM-101 exposure after repeated 2.5 mg/kg SC administrations in humans using different dosing schedules. Serum concentrations vs. time data, linear scale.

FIG. 5 depicts simulated CM-101 exposure after repeated 5 mg/kg SC administrations in humans using different dosing schedules. Serum concentrations vs. time data, linear scale.

FIGS. 6A-6E show fold change of total CCL24 levels in healthy volunteers treated with a single ascending dose of CM-101 compared to placebo. FIG. 6A shows Cohort 1-administration of 0.75 mg/kg CM-101 compared to placebo. FIG. 6B shows Cohort 2-administration of 2.5 mg/kg CM-101 compared to placebo. FIG. 6C shows Cohort 3-administration of 5 mg/kg CM-101 compared to placebo. FIG. 6D shows Cohort 4-administration of 10 mg/kg CM-101 compared to placebo. FIG. 6E shows Placebo, Cohort 2, 3 and 4.

FIG. 7 shows fold change of total CCL24 levels in healthy volunteers treated with a single injection of 5 mg/kg CM-101 SC compared to placebo. Total levels of CCL24 are presented as change from baseline (percentage).

FIG. 8 shows fold change of total CCL24 levels in healthy volunteers treated with a single dose of 5 mg/kg CM-101 using IV or SC administration. Single administration of 5 mg/kg CM-101 was given by either infusion (IV) or injection (SC). Total levels of CCL24 are presented as change from baseline (percentage).

FIG. 9 shows PK-PD correlation in healthy volunteers treated with a single 5 mg/kg SC dose of CM-101. Single administration of 5 mg/kg CM-101 was given by injection (SC). CM-101 levels, plotted on the right Y axis, were measured using a validated assay. Total levels of CCL24 are presented in change from baseline (percentage), plotted on the left Y-axis.

FIG. 10 shows the pharmacokinetics of CM-101 in NAFLD patients treated with five repeated doses of 2.5 mg/kg IV or 5 mg/kg SC CM101. The graph presents CM-101 levels (μg/ml) in the serum as a function of time (days).

FIG. 11 shows the levels of collagen turnover markers in NAFLD patients treated with CM101 or placebo. The graph presents relative change from baseline (%) for each of the collagen turnover markers: Pro-C3, Pro-C4 and C3M. Results are shown for patients that had baseline elastography (as measured by FibroScan) that was >4 kPA (n=10 CM-101; n=3 placebo).

FIG. 12 shows the levels of serum fibrotic biomarkers in NAFLD patients treated with CM101 or placebo. The graph presents relative change from baseline (%) for each of the serum fibrotic biomarkers: TIMP-1, TIMP-2 and PDGF AA. Results are shown for patients that had baseline elastography (as measured by FibroScan) that was >4 kPA (n=10 CM-101; n=3 placebo).

FIG. 13 shows the elastography in NAFLD patients treated with CM101 or placebo. The graph presents liver stiffness (relative change from baseline (%)). Results are shown for patients that had baseline elastography (as measured by FibroScan) that was >4 kPA (n=10 CM-101; n=3 placebo).

### DETAILED DESCRIPTION

As disclosed herein, fibrotic inflammatory diseases, such as non-alcoholic steatohepatitis (NASH), primary sclerosing cholangitis (PSC) and Systemic sclerosis (SSc), can be treated by subcutaneous or intravenous administration of an anti-CCL24 monoclonal antibody. In one aspect, the present disclosure provides doses, routes and schedules of administering an anti-CCL24 monoclonal antibody (e.g., CM-101) that can effectively modulate signaling of CCL24 in a human subject having a fibrotic inflammatory disease and achieve favorable clinical outcomes as measured by one or more tests as described herein. Also disclosed are the pharmacokinetics and pharmacodynamics of the anti-CCL24 monoclonal antibody (e.g., CM-101) according to such administrations that lead to desirable clinical outcomes.

In one aspect, the present disclosure provides methods of treating fibrotic inflammatory diseases in a human subject, comprising administering an effective amount of an anti-CCL24 monoclonal antibody or a pharmaceutical composition thereof. In some embodiments, the present disclosure provides an anti-CCL24 monoclonal antibody, or a pharmaceutical composition thereof, for use in the treatment of fibrotic inflammatory diseases.

In some embodiments, the fibrotic inflammatory diseases are selected from the group consisting of non-alcoholic fatty liver diseases (NAFLD), cholestasis, intrahepatic cholestatic liver diseases, hepatitis (e.g., alcoholic hepatitis), liver cirrhosis and systemic sclerosis (SSc). In some embodiments, said NAFLD is nonalcoholic fatty liver (NAFL) or nonalcoholic steatohepatitis (NASH). In some embodiments, said hepatic pathology is Hepatocellular carcinoma resulting from NASH. In some embodiments, said intrahepatic cholestatic liver disease is primary sclerosing cholangitis (PSC) or primary biliary cirrhosis (PBC). In some embodiments, said hepatic pathology is cholangiocarcinoma resulting from PSC.

In some embodiments, the fibrotic inflammatory disease is nonalcoholic steatohepatitis (NASH). In some embodiments, the fibrotic inflammatory disease is primary sclerosing cholangitis (PSC). In some embodiments, the fibrotic inflammatory disease is systemic sclerosis (SSC).

In one aspect, the disclosure relates to an anti-CCL24 antibody, or a pharmaceutical composition therefore, for use in:

a) reducing elevated serum levels of liver enzymes upon liver damage;

b) reducing liver damage, necrosis or fibrosis;

c) attenuating the transition of hepatic stellate cells (HSC) to myofibroblasts; and/or

d) reducing or inhibiting CCR3 activation in a cell;

In some embodiments, the anti-CCL24 antibody may be a monoclonal antibody. In some embodiments, the anti-CCL24 antibody may be a chimeric antibody, a human antibody, a humanized antibody, or an antigen-binding fragment thereof selected from the group consisting of Fv, single chain Fv (scFv), heavy chain variable region capable of binding the antigen, light chain variable region capable of binding the antigen, Fab, F(ab)2′ and any combination thereof. Anti-CCL24 antibodies are described, e.g., WO 2010/086854, WO 2015/132790, and WO2018/163185, each of which is incorporated herein by reference in its entirety.

In some embodiments, the anti-CCL24 antibody is a humanized monoclonal antibody CM-101 as described herein.

### Anti-CCL24 Monoclonal Antibody

In some embodiments, the anti-CCL24 monoclonal antibody is a humanized monoclonal antibody or an antigen-binding fragment thereof, wherein said antibody comprises:
a heavy chain variable region comprising:
a) the complementary determining region VH CDR1 comprising the amino acid sequence denoted by SEQ ID NO: 1;
b) the complementary determining region VH CDR2 comprising the amino acid sequence denoted by SEQ ID NO: 2; and
c) the complementary determining region VH CDR3 comprising the amino acid sequence denoted by SEQ ID NO: 3; and
a light chain variable region comprising:
d) the complementary determining region VK CDR1 comprising the amino acid sequence denoted by SEQ ID NO: 4;
e) the complementary determining region VK CDR2 comprising the amino acid sequence denoted by SEQ ID NO: 5; and
f) the complementary determining region VK CDR3 comprising the amino acid sequence denoted by SEQ ID NO: 6.

In accordance with the disclosure VH CDR1 comprises the amino acid sequence NSGMN denoted by SEQ ID NO: 1; VH CDR2 comprises the amino acid sequence WINTYNGEPTYTDDFKG denoted by SEQ ID NO: 2, VH CDR3 comprises the amino acid sequence HSYGSSYAMDN denoted by SEQ ID NO: 3; VK CDR1 comprises the amino acid sequence KASQSVDYDGDSYMN denoted by SEQ ID NO: 4; VK CDR2 comprises the amino acid sequence VASNLKS denoted by SEQ ID NO: 5; and VK CDR3 comprises the amino acid sequence QQSNEEPWT denoted by SEQ ID NO: 6. In one specific embodiment, the present disclosure provides an anti-CCL24 monoclonal antibody, or an antigen-binding fragment thereof, comprising the complementary determining region VH CDR3 comprising the amino acid sequence HSYGSSYAMDN denoted by SEQ ID NO: 3, for use in the treatment of hepatic pathologies.

The above CDR sequences CDRH1, CDRH2, CDRH3, CDRL1, CDRL2 and CDRL3 denoted by SEQ ID NO: 1, SEQ ID NO: 2, SEQ ID NO: 3, SEQ ID NO: 4, SEQ ID NO: 5 and SEQ ID NO: 6, respectively are also presented in the context of their respective heavy and light chains sequences.

In some embodiments, the anti-CCL24 monoclonal antibody or an antigen binding fragment thereof comprises a heavy chain variable region of SEQ ID NO: 7:

QIQLVQSGPELKKPGASVKVSCRASGYPFTNSGMNWVKQAPGKGLKWMG
WINTYNGEPTYTDDFKGRFAFSLETSASTAYLQINNLRNEDTATYFCAS
HSYGSSYAMDNWGQGTSVTVSS

In some embodiments, the anti-CCL24 monoclonal antibody or an antigen binding fragment thereof comprises a light chain variable region of SEQ ID NO: 8:

DIVLTQSPDSLAVSLGERATINCKASQSVDYDGDSYMNWYQQKPGQPPK
LLIYVASNLKSGIPARFSGSGSGTDFTLTISSLQPEDFATYYCQQSNEE
PWTFGGGTKVEIK

In some embodiments, the anti-CCL24 monoclonal antibody comprises the heavy chain variable region of SEQ ID NO:7 and the light chain variable region of SEQ ID NO: 8.

In some embodiments, the anti-CCL24 monoclonal antibody according to the disclosure is a humanized monoclonal antibody comprising the heavy chain variable region denoted by SEQ ID NO:7 with deletion and/or substitution of 0, 1, 2, 3, 4, 5, 6, 7, 8, 9, or 10 residues at its N- and/or C-terminus, and/or the light chain variable region denoted by SEQ ID NO: 8 with deletion and/or substitution of 0, 1, 2, 3, 4, 5, 6, 7, 8, 9, or 10 residues at its N- and/or C-terminus. In some embodiments, the anti-CCL24 monoclonal antibody comprises a heavy chain variable region having at least 80%, at least 85%, at least 90%, at least 95%, at least 98%, at least 99%, or 100% sequence identity to SEQ ID NO:7 and/or a light chain variable region having at least 80%, at least 85%, at least 90%, at least 95%, at least 98%, at least 99%, or 100% sequence identity to SEQ ID NO: 8.

In some embodiments, the anti-CCL24 monoclonal antibody according to the disclosure is a humanized monoclonal antibody comprising six CDR sequences as denoted by SEQ ID Nos 1-6, and a heavy chain variable region having at least 80%, at least 85%, at least 90%, at least 95%, at least 98%, at least 99%, or 100% sequence identity to SEQ ID NO:7, and/or a light chain variable region having at least 80%, at least 85%, at least 90%, at least 95%, at least 98%, at least 99%, or 100% sequence identity to SEQ ID NO: 8.

In some embodiments, the present disclosure provides an anti-CCL24 monoclonal antibody that binds the same epitope as an antibody comprising:

(a) a heavy chain CDR1 comprising NSGMN (SEQ ID NO: 1), a heavy chain CDR2 comprising WINTYNGEPTYTDDFKG (SEQ ID NO: 2), and a heavy chain CDR3 comprising HSYGSSYAMDN (SEQ ID NO: 3); and

(b) a light chain CDR1 comprising KASQSVDYDGDSYMN (SEQ ID NO: 4), a light chain CDR2 comprising VASNLKS (SEQ ID NO: 5), and a light chain CDR3 comprising QQSNEEPWT (SEQ ID NO: 6).

In some embodiments, the anti-CCL24 monoclonal antibody for use according to the disclosure is a humanized monoclonal antibody, wherein said antibody comprises a heavy chain variable region encoded by a nucleic acid sequence according to SEQ ID NO: 9 and a light chain variable region encoded by a nucleic acid sequence according to SEQ ID NO: 10. In some embodiments, the anti-CCL24 monoclonal antibody comprises a heavy chain variable region encoded by a nucleic acid having at least 80%, at least 85%, at least 90%, at least 95%, at least 98%, at least 99%, or 100% sequence identity to SEQ ID NO: 9 and/or a light chain variable region encoded by a nucleic acid having at least 80%, at least 85%, at least 90%, at least 95%, at least 98%, at least 99%, or 100% sequence identity to SEQ ID NO: 10.

In some embodiments, the anti-CCL24 monoclonal antibody for use according to the disclosure is a humanized monoclonal antibody termed CM-101, wherein said antibody comprises a heavy chain according to SEQ ID NO: 11 and a light chain according to SEQ ID NO: 12:

(SEQ ID NO: 11)
QIQLVQSGPELKKPGASVKVSCRASGYPFTNSGMNWVKQAPGKGLKWMG
WINTYNGEPTYTDDFKGRFAFSLETSASTAYLQINNLRNEDTATYFCAS
HSYGSSYAMDNWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
(SEQ ID NO: 12)
DIVLTQSPDSLAVSLGERATINCKASQSVDYDGDSYMNWYQQKPGQPPK
LLIYVASNLKSGIPARFSGSGSGTDFTLTISSLQPEDFATYYCQQSNEE
PWTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

In some embodiments, the anti-CCL24 monoclonal antibody according to the disclosure is a humanized monoclonal antibody comprising the heavy chain denoted by SEQ ID NO: 11 with amino acid variation of 0, 1, 2, 3, 4, 5, 6, 7, 8, 9, or 10 residues, and/or the light chain denoted by SEQ ID NO: 12 with amino acid variation of 0, 1, 2, 3, 4, 5, 6, 7, 8, 9, or 10 residues. In some embodiments, the anti-CCL24 monoclonal antibody comprises a heavy chain having at least 80%, at least 85%, at least 90%, at least 95%, at least 98%, at least 99%, or 100% sequence identity to SEQ ID NO: 11 and/or a light chain having at least 80%, at least 85%, at least 90%, at least 95%, at least 98%, at least 99%, or 100% sequence identity to SEQ ID NO: 12.

In some embodiments, the anti-CCL24 monoclonal antibody is administered in combination with at least one additional therapeutic agent. In some embodiments, said at least one additional therapeutic agent is selected from a group consisting of chemotherapeutics, cytokines, peptides, antibodies and antibiotics. In certain embodiments, said additional therapeutic agent includes, but is not limited to farnesoid X receptor (FXR) agonists (e.g., cafestol, chenodeoxycholic acid, obeticholic acid, fexaramine), peroxisome proliferator-activated receptor (PPAR) agonists, anti-chemokine or cytokine monoclonal antibodies, small molecules which inhibit chemokine or cytokine functions, anti-inflammatory and anti-fibrotic agents, including steroids and soluble protein anti-inflammatory agents. In some embodiments, the additional therapeutic agent is an additional antibody. In some embodiments, said at least one additional therapeutic agent is selected from the group consisting of farnesoid X receptor (FXR) agonists, peroxisome proliferator-activated receptor (PPAR) agonists, anti-chemokine or anti-cytokine monoclonal antibodies, small molecules, anti-inflammatory agents, anti-fibrotic agents, and steroids. In some embodiments, the anti-CCL24 monoclonal antibody or a pharmaceutical composition therefore is administered prior to, concomitantly or subsequently to the administration of said at least one additional therapeutic agent. In some embodiments, said pharmaceutical composition further comprises at least one additional therapeutic agent.

In some embodiments, the antibody of the disclosure is a monoclonal antibody. The term “monoclonal antibody”, “monoclonal antibodies” or “mAb” as herein defined refers to a population of substantially homogenous antibodies, i.e., the individual antibodies comprising the population are identical except for possibly naturally occurring mutations that may be present in minor amounts. Monoclonal antibodies are directed against a single antigenic site.

An exemplary antibody structural unit comprises a tetramer, as known in the art. Each tetramer is composed of two identical pairs of polypeptide chains, each pair having one “light chain” and one “heavy chain”. The N-terminus of each chain defines a variable region of about 100 to 110 or more amino acids primarily responsible for antigen (or epitope) recognition.

Thus, the terms “heavy chain variable region” (VH) and “light chain variable region” (VL) refer to these heavy and light chains, respectively. More specifically, the variable region is subdivided into hypervariable and framework (FR) regions. Hypervariable regions have a high ratio of different amino acids in a given position, relative to the most common amino acid in that position. Four FR regions which have more stable amino acids sequences separate the hypervariable regions. The hypervariable regions directly contact a portion of the antigen's surface. For this reason, hypervariable regions are herein referred to as “complementarity determining regions”, or “CDRs”.

From N-terminal to C-terminal, both light and heavy chains comprise the domains FR1, CDR1, FR2, CDR2, FR3, CDR3 and FR4. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located.

Thus, the complementarity determining regions CDRH1, CDRH2 and CDRH3 refer to the three complementarity determining regions starting from the N-terminus of the antibody's heavy chain and the complementarity determining regions CDRL1, CDRL2 and CDRL3 refer to the three complementarity determining regions starting from the N-terminus of the antibody's light chain. In some embodiments the anti CCL24 monoclonal antibody is a chimeric antibody, a human antibody, a humanized antibody or an antigen binding fragment thereof.

The term “chimeric” antibodies as herein defined refers to antibodies in which a portion of the heavy and/or light chain is derived from a particular species, while the remainder of the chain(s) is derived from another species.

The term “humanized” antibodies traditionally refers to forms of non-human (for example, murine) antibodies that contain a human-derived immunoglobulin framework with minimal sequences derived from non-human immunoglobulin at the CDRs and optionally at additional relevant positions. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and activity. In some embodiments, the humanized antibodies of the present disclosure were prepared using the Composite Human Antibodies™ technology that minimizes immunogenicity of the antibodies in patients. In this humanization technology multiple sequence segments derived from variable (V) regions of unrelated human antibodies are used as acceptors for the complementarity determining regions (CDRs) of the starting antibodies. Through careful selection of human sequence segments and the application of in silico tools, CD4+ T cell epitopes are avoided so the risk of immunogenicity is reduced compared to standard humanized antibodies whilst antibody affinity and specificity is maintained.

The term “human antibody” as used herein refers to an antibody that possesses an amino acid sequence corresponding to that of an antibody produced by a human and/or has been made using any of the techniques for making human antibodies known in the art. This definition specifically excludes a humanized antibody that comprises non-human antigen-binding residues.

For preparing large quantities of the antibody (either chimeric, humanized, fully humanized or human), a stable cell line expressing the antibody can be prepared, by transfecting cells (e.g., CHO cells) with the Ig expression vector containing both heavy and light chains of the antibody. The antibodies may then be manufactured in a state of the art single-use bioreactor system. The antibodies may be purified to clinical grade using well established monoclonal antibody purification methods. Highly anti-CCL24 antibody producing clones may be then selected and expanded based on antibody levels in the supernatant, as tested by any method known in the art, for example, an CCL24-specific ELISA assay, as detailed herein below. A master cell bank, developed for the specific clone, may serve as the starting growing material for all clinical grade batches.

The present disclosure also encompasses variants of the heavy and light chain variable regions. The variants may include mutations in the complementarity determining regions of the heavy and light chains which do not alter the activity of the antibodies herein described, or in the framework region.

By the term “variant” it is meant sequences of amino acids or nucleotides different from the sequences specifically identified herein, in which one or more amino acid residues or nucleotides are deleted, substituted or added.

Variants encompass various amino acid substitutions. An amino acid “substitution” is the result of replacing one amino acid with another amino acid which has similar or different structural and/or chemical properties. Amino acid substitutions may be made on the basis of similarity in polarity, charge, solubility, hydrophobicity, hydrophilicity, and/or the amphipathic nature of the residues involved.

Typically, variants encompass conservative amino acid substitutions. Conservative substitution tables providing functionally similar amino acids are well known in the art. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Each of the following eight groups contains other exemplary amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M).

Conservative nucleic acid substitutions are nucleic acid substitutions resulting in conservative amino acid substitutions as defined above.

Variants in accordance with the disclosure also encompass non-polar to polar amino acid substitutions and vice-versa.

As used herein, the term “amino acid” or “amino acid residue” refers to naturally occurring and synthetic amino acids, as well as amino acid analogs and amino acid mimetics that function in a manner similar to the naturally occurring amino acids.

Variant sequences refer to amino acid or nucleic acids sequences that may be characterized by the percentage of the identity of their amino acid or nucleotide sequences with the amino acid or nucleotide sequences described herein (for example, the amino acid or nucleotide sequences of the heavy and light chains of the antibodies herein described).

In some embodiments, variant sequences as herein defined refer to nucleic acid sequences that encode the heavy and light chain variable regions, each having a sequence of nucleotides with at least 70% or 75% of sequence identity, around 80% or 85% of sequence identity, around 90%, 91%, 92%, 93%, 94%, 95%, 96%, 97%, 98% or 99% of sequence identity when compared to the sequences of the heavy and light chain variable regions described herein.

In some embodiments, the present disclosure provides immunoconjugates comprising an anti-CCL24 monoclonal antibody as described herein and an additional therapeutic agent as defined herein below.

### Fibrotic Inflammatory Diseases

In one aspect, the disclosure provides methods of prophylaxis, treatment or amelioration of fibrotic inflammatory diseases, comprising administering to a subject in need thereof an effective amount of the anti-CCL24 monoclonal antibody or a pharmaceutical composition according to the disclosure. In some embodiments, the disclosure provides an anti-CCL24 monoclonal antibody or a pharmaceutical composition therefore for use in a method of prophylaxis, treatment or amelioration of a fibrotic inflammatory disease as described herein.

In some embodiments, the present disclosure provides methods of treating primary sclerosing cholangitis (PSC) in a human subject, comprising administering an effective amount of an anti-CCL24 monoclonal antibody or a pharmaceutical composition thereof. In some embodiments, the anti-CCL24 monoclonal antibody is CM-101.

In some embodiments, the present disclosure provides methods of treating nonalcoholic steatohepatitis (NASH) in a human subject, comprising administering an effective amount of an anti-CCL24 monoclonal antibody or a pharmaceutical composition thereof. In some embodiments, the anti-CCL24 monoclonal antibody is CM-101.

In some embodiments, the present disclosure provides methods of treating systemic sclerosis (SSc) in a human subject, comprising administering an effective amount of an anti-CCL24 monoclonal antibody or a pharmaceutical composition thereof. In some embodiments, the anti-CCL24 monoclonal antibody is CM-101.

Also provided are methods of reducing elevated serum levels of liver enzymes (e.g., ALT, AST, alkaline phosphatase and bilirubin), comprising administering to a subject in need thereof an effective amount of the anti-CCL24 monoclonal antibody or a pharmaceutical composition thereof according to the disclosure. Also provided are methods of reducing liver damage, inflammation or fibrosis, comprising administering to a subject in need thereof an effective amount of the anti-CCL24 monoclonal antibody or a pharmaceutical composition thereof according to the disclosure. Also provided are methods of attenuating the transition of hepatic stellate cells (HSC) to myofibroblasts, comprising administering to a subject in need thereof an effective amount of the anti-CCL24 monoclonal antibody or a pharmaceutical composition thereof according to the disclosure. In some embodiments, the anti-CCL24 monoclonal antibody is CM-101.

An “effective amount” of the anti-CCL24 monoclonal antibody according to the disclosure, or the pharmaceutical composition according thereof, is determined by such considerations in order to be sufficient to cure, arrest or at least alleviate or ameliorate at least one clinical symptom of the medical condition.

### Fibrotic Inflammatory Diseases Treatment

In some embodiments, administration of the anti-CCL24 monoclonal antibody or a pharmaceutical composition thereof increases total sera level of CCL24 compared to baseline total sera level of CCL24 before administering the anti-CCL24 monoclonal antibody. In some embodiments, administration of the anti-CCL24 monoclonal antibody or a pharmaceutical composition thereof increases total sera level of CCL24 to at least 125%, at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured at about 8 hour, about 16 hour or about 24 hour post administration of the anti-CCL24 monoclonal antibody. In some embodiments, the increase of total sera level of CCL24 is measured at day 1, day 2, day 3, day 4, day 5, day 6, day 7, day 8, day 9, day 10 or day 11 post administration. In some embodiments, subcutaneous administration of 5 mg/kg of the anti-CCL24 monoclonal antibody increases total sera level of CCL24 at day 4 post administration to at least 150%, at least 175%, at least 200%, at least 250% or at least 300% of baseline total sera level of CCL24 before administering the anti-CCL24 monoclonal antibody. In some embodiments, intravenous administration of 10 mg/kg of the anti-CCL24 monoclonal antibody increases total sera level of CCL24 at day 4 post administration to at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured between the first and the second administrations of the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof improves hepatic condition of the subject as evaluated by one or more circulating fibrosis markers. In some embodiments, administration of the anti-CCL24 monoclonal antibody or a pharmaceutical composition thereof lowers the level of one or more fibrosis or inflammatory markers compared to baseline before administering the anti-CCL24 monoclonal antibody. Fibrosis or inflammatory markers include plasma alanine aminotransferase (ALT), alkaline phosphatase (ALP), aspartate aminotransferase (AST), PRO-C3, PRO-C4, C3M (PRO-C3, PRO-C4 and C3M are also referred to herein as collagen turnover markers), PRO-05, cytokeratin 18 (CK18), amino-terminal propeptide of type III procollagen (PIIINP), hyaluronic acid (HA), Tissue Inhibitor of Metalloproteinases 1 (TIMP-1), Tissue Inhibitor of Metalloproteinases 2 (TIMP-2), platelet derived growth factor (PDGF) (e.g. PDGF AA), γ-glutamyltransferase (GGT), bilirubin, C-reactive protein (CRP), erythrocyte sedimentation rate (ESR), and procalcitonin (PCT). Levels in the markers can be tested by well-established methods, for example, ELISA assay, or methods described in the examples herein.

In some embodiments, administration of the anti-CCL24 monoclonal antibody or a pharmaceutical composition thereof lowers the level of circulating alkaline phosphatase (ALP) compared to baseline before administering the anti-CCL24 monoclonal antibody. In some embodiments, administration of the anti-CCL24 monoclonal antibody or a pharmaceutical composition thereof lowers the level of circulating alkaline phosphatase (ALP) by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of circulating alkaline phosphatase (ALP) by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of γ-glutamyltransferase (GGT) compared to baseline before administering the anti-CCL24 monoclonal antibody. In some embodiments, administration of the anti-CCL24 monoclonal antibody or a pharmaceutical composition thereof lowers the level of γ-glutamyltransferase (GGT) by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of γ-glutamyltransferase (GGT) by about 5% to about 30%, about 20% to about 50%, about 40% to about 70%, or about 60% to about 99% compared to baseline before administering the anti-CCL24 monoclonal antibody. In some embodiments, administration of the anti-CCL24 monoclonal antibody or a pharmaceutical composition thereof lowers the level of γ-glutamyltransferase (GGT) by at least 20% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of alanine aminotransferase (ALT) compared to baseline before administering the anti-CCL24 monoclonal antibody. In some embodiments, administration of the anti-CCL24 monoclonal antibody or a pharmaceutical composition thereof lowers the level of alanine aminotransferase (ALT) by at least about 1%, at least about 2.5%, at least about 5%, at least about 7.5%, at least about 10%, at least about 12%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of alanine aminotransferase (ALT) by about 5% to about 20%, about 10% to about 30%, about 20% to about 50%, about 40% to about 70%, or about 60% to about 99% compared to baseline before administering the anti-CCL24 monoclonal antibody. In some embodiments, administration of the anti-CCL24 monoclonal antibody or a pharmaceutical composition thereof lowers the level of alanine aminotransferase (ALT) by at least 10% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating PRO-C3 (N-terminal pro-peptide of type III collagen (neo-peptide)) compared to baseline before administering the anti-CCL24 monoclonal antibody. In some embodiments, administration of the anti-CCL24 monoclonal antibody or a pharmaceutical composition thereof lowers the level of PRO-C3 by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of PRO-C3 by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating PRO-C4 (representing the internal epitope in 7S domain of type IV collagen) compared to baseline before administering the anti-CCL24 monoclonal antibody. In some embodiments, administration of the anti-CCL24 monoclonal antibody or a pharmaceutical composition thereof lowers the level of PRO-C4 by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of PRO-C4 by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating C3M (a neo-epitope of MMP-9 mediated degradation of type III collagen that reflects the liver inflammatory state) compared to baseline before administering the anti-CCL24 monoclonal antibody. In some embodiments, administration of the anti-CCL24 monoclonal antibody or a pharmaceutical composition thereof lowers the level of C3M by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of C3M by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating PRO-05 (pro-peptide of type V collagen) compared to baseline before administering the anti-CCL24 monoclonal antibody. In some embodiments, administration of the anti-CCL24 monoclonal antibody or a pharmaceutical composition thereof lowers the level of PRO-05 by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of PRO-05 by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers Enhanced Liver Fibrosis (ELF) score compared to baseline before administering the anti-CCL24 monoclonal antibody. In some embodiments, administration of the anti-CCL24 monoclonal antibody or a pharmaceutical composition thereof lowers Enhanced Liver Fibrosis (ELF) score by at least about 0.1, at least about 0.2, at least about 0.3, at least about 0.5, at least about 0.7, at least about 1.0, at least about 1.5, at least about 2, at least about 3, at least about 4, at least about 5, or more than 5, compared to baseline before administering the anti-CCL24 monoclonal antibody. The Enhanced Liver Fibrosis (ELF) test is a non-invasive blood test that measures three direct markers of fibrosis: hyaluronic acid (HA), procollagen III amino-terminal peptide (PIIINP), and tissue inhibitor of matrix metalloproteinase 1 (TIMP-1). The ELF test, in conjunction with other laboratory and clinical findings, can be used to assess the risk of progression to cirrhosis and LREs in patients with chronic liver disease. In some embodiments, an ELF score of <7.7 indicates no to mild fibrosis, an ELF score of 7.7 to 9.8 indicates moderate fibrosis, an ELF score of 9.8 to 11.3 indicates severe fibrosis, and an ELF score of >=11.3 indicates cirrhosis.

In some embodiments, administration of the anti-CCL24 monoclonal antibody or a pharmaceutical composition thereof lowers Enhanced Liver Fibrosis (ELF) score by at least 0.1, at least 0.2, at least 0.3, at least 0.5, at least 0.7, at least 0.8, at least 1.0, at least 1.2, at least 1.5, at least 2.0, at least 2.5, at least 3.0, at least 3.5, at least 4.0, at least 5.0, at least 6.0, at least 7.0, at least 8.0, or at least 9.0 compared to baseline before administering the anti-CCL24 monoclonal antibody. In some embodiments, administration of the anti-CCL24 monoclonal antibody or a pharmaceutical composition thereof lowers Enhanced Liver Fibrosis (ELF) score by about 0.1 to about 1, about 0.5 to about 1.5, about 1.0 to about 3.0, about 1.5 to about 4.0, or about 2.0 to about 6.0 compared to baseline before administering the anti-CCL24 monoclonal antibody. In some embodiments, administration of the anti-CCL24 monoclonal antibody or a pharmaceutical composition thereof lowers Enhanced Liver Fibrosis (ELF) score to less than 11.5, less than 11.0, less than 10.5, less than 10.0, less than 9.5, less than 9.0, less than 8.5, less than 8.0, less than 7.5, or less than 7.0. In some embodiments, the anti-CCL24 monoclonal antibody is CM-101.

In some embodiments, administration of the anti-CCL24 monoclonal antibody or a pharmaceutical composition thereof improves hepatic condition as evaluated by one or more imaging tests. In some embodiments, the imaging test is liver elastography. In some embodiments, the imaging test is an ultrasound elastography such as FibroScan. In some embodiments, the imaging test is magnetic resonance imaging. In some embodiments, the imaging test is Magnetic Resonance Imaging Proton Density Fat Fraction (MRI-PDFF). In some embodiments, the hepatic condition is liver fat content. In some embodiments, liver fat content is measured by MRI-PDFF, FibroScan, or other liver elastography and/or imaging tests.

In some embodiments, administration of the anti-CCL24 monoclonal antibody or a pharmaceutical composition thereof lowers the level of liver fat content compared to baseline before administering the anti-CCL24 monoclonal antibody. In some embodiments, administration of the anti-CCL24 monoclonal antibody or a pharmaceutical composition thereof lowers the level of liver fat content by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of liver fat content by about 5% to about 20%, about 10% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101. In some embodiments, the liver fat content is evaluated by Magnetic Resonance Imaging such as MRI-PDFF. In some embodiments, the liver fat content is measured by FibroScan CAP score.

FibroScan can provide fibrosis result which is a measurement of the amount of scarring in the liver. FibroScan measures scarring by measuring the stiffness of the liver. The fibrosis result is measured in kilopascals (kPa). In some embodiments, a range of 2 to 7 kPa indicates no liver scarring or mild liver scarring, a range of 7.5 to 10 kPa indicates moderate liver scarring, a range of 10 to 14 kPa indicates severe liver scarring, and a range of 14 kPa or higher indicates advanced liver scarring (cirrhosis). FibroScan can also provide CAP score which is a measurement of fatty change or fat content in the liver. The CAP score is measured in decibels per meter (dB/m). Typically, it ranges from 100 to 400 dB/m.

In some embodiments, administration of the anti-CCL24 monoclonal antibody or a pharmaceutical composition thereof decreases liver stiffness compared to baseline before administering the anti-CCL24 monoclonal antibody. In some embodiments, the liver stiffness is measured by FibroScan. In some embodiments, administration of the anti-CCL24 monoclonal antibody or a pharmaceutical composition thereof decreases liver stiffness, as measured by FibroScan fibrosis result, by at least 0.1 kPa, at least 0.2 kPa, at least 0.3 kPa, at least 0.5 kPa, at least 0.7 kPa, at least 1.0 kPa, at least 1.2 kPa, at least 1.5 kPa, at least 2.0 kPa, at least 2.5 kPa, at least 3.0 kPa, at least 3.5 kPa, at least 4.0 kPa, at least 5.0 kPa, at least 6.0 kPa, at least 8.0 kPa, at least 10 kPa, at least 15 kPa, at least 20 kPa, at least 25 kPa, or at least 30 kPa, compared to baseline before administering the anti-CCL24 monoclonal antibody. In some embodiments, administration of the anti-CCL24 monoclonal antibody or a pharmaceutical composition thereof decreases liver stiffness, as measured by FibroScan fibrosis result, by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases CAP score, as measured by FibroScan, by at least 5 dB/m, at least 10 dB/m, at least 15 dB/m, at least 20 dB/m, at least 30 dB/m, at least 40 dB/m, at least 50 dB/m, at least 60 dB/m, at least 80 dB/m, at least 100 dB/m, at least 120 dB/m, at least 150 dB/m, or at least 200 dB/m compared to baseline before administering the anti-CCL24 monoclonal antibody. In some embodiments, administration of the anti-CCL24 monoclonal antibody or a pharmaceutical composition thereof decreases CAP score, as measured by FibroScan, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases serum level of Tissue Inhibitor of Metalloproteinases 1 (TIMP-1) compared to baseline before administering the anti-CCL24 monoclonal antibody. In some embodiments, administration of the anti-CCL24 monoclonal antibody or a pharmaceutical composition thereof decreases serum level of Tissue Inhibitor of Metalloproteinases 1 (TIMP-1) by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 15%, at least about 17%, at least about 20%, at least about 25%, at least about 30%, at least about 35%, at least about 40%, at least about 50%, at least about 60%, at least about 70%, at least about 80%, or at least about 90%, compared to baseline before administering the anti-CCL24 monoclonal antibody. In some embodiments, administration of the anti-CCL24 monoclonal antibody or a pharmaceutical composition thereof decreases serum level of Tissue Inhibitor of Metalloproteinases 1 (TIMP-1) by at least about 10% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases serum level of Tissue Inhibitor of Metalloproteinases 2 (TIMP-2) compared to baseline before administering the anti-CCL24 monoclonal antibody. In some embodiments, administration of the anti-CCL24 monoclonal antibody or a pharmaceutical composition thereof decreases serum level of Tissue Inhibitor of Metalloproteinases 2 (TIMP-2) by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 15%, at least about 17%, at least about 20%, at least about 25%, at least about 30%, at least about 35%, at least about 40%, at least about 50%, at least about 60%, at least about 70%, at least about 80%, or at least about 90%, compared to baseline before administering the anti-CCL24 monoclonal antibody. In some embodiments, administration of the anti-CCL24 monoclonal antibody or a pharmaceutical composition thereof decreases serum level of Tissue Inhibitor of Metalloproteinases 2 (TIMP-2) by at least about 5% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases serum level of platelet derived growth factor (PDGF), e.g., PDGF AA, compared to baseline before administering the anti-CCL24 monoclonal antibody. In some embodiments, administration of the anti-CCL24 monoclonal antibody or a pharmaceutical composition thereof decreases serum level of PDGF by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 15%, at least about 17%, at least about 20%, at least about 25%, at least about 30%, at least about 35%, at least about 40%, at least about 50%, at least about 60%, at least about 70%, at least about 80%, or at least about 90%, compared to baseline before administering the anti-CCL24 monoclonal antibody. In some embodiments, administration of the anti-CCL24 monoclonal antibody or a pharmaceutical composition thereof decreases serum level of PDGF AA by at least about 5% or at least about 10% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases NAFLD activity score (NAS score), as measured by liver biopsy, by at least 1 point, at least 2 points, at least 3 points, at least 4 points, at least 5 points, at least 6 points, at least 7 points compared to baseline before administering the anti-CCL24 monoclonal antibody. In some embodiments, administration of the anti-CCL24 monoclonal antibody or a pharmaceutical composition thereof decreases NAS score, as measured by liver biopsy, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases fibrosis score, as measured by liver biopsy, by at least 1 point, at least 2 points, at least 3 points, at least 4 points compared to baseline before administering the anti-CCL24 monoclonal antibody. In some embodiments, administration of the anti-CCL24 monoclonal antibody or a pharmaceutical composition thereof decreases fibrosis score, as measured by liver biopsy, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof improves Combined Response Index for Systemic Sclerosis (CRISS) score of the subject. The calculation of CRISS is a 2-step process for use in a clinical trial and is described in Khanna et. al., Arthritis Rheumatol. 2016 February; 68(2):299-311. In some embodiments, subjects for whom the predicted probability is >=0.60 are considered improved, while subjects for whom the predicted probability is <0.60 are considered not improved. In some embodiments, the administration of the anti-CCL24 monoclonal antibody or a pharmaceutical composition thereof results in an average CRISS score of about 0.1, about 0.2, about 0.1, about 0.3, about 0.4, about 0.5, about 0.6, about 0.7, about 0.8, about 0.9, or about 1.0. In some embodiments, the administration of the anti-CCL24 monoclonal antibody or a pharmaceutical composition thereof results in an average CRISS score of at least 0.1, at least 0.2, at least 0.1, at least 0.3, at least 0.4, at least 0.5, at least 0.6, at least 0.7, at least 0.8, at least 0.9, or at least 1.0. In some embodiments, administration of the anti-CCL24 monoclonal antibody or a pharmaceutical composition improves lung function of the subject compared to baseline before administering the anti-CCL24 monoclonal antibody in subjects suffering from Systemic Sclerosis (SSc). In some embodiments, the anti-CCL24 monoclonal antibody is CM-101.

In some embodiments, administration of the anti-CCL24 monoclonal antibody or a pharmaceutical composition thereof improves lung function of the subject compared to baseline before administering the anti-CCL24 monoclonal antibody. In some embodiments, administration of the anti-CCL24 monoclonal antibody or a pharmaceutical composition thereof improves lung function of the subject by at least about 5%, at least about 10%, at least about 20%, at least about 30%, at least about 40%, at least about 50%, at least about 60%, at least about 70%, at least about 80%, at least about 90%, at least about 100%, at least about 150% or at least about 200%, compared to baseline before administering the anti-CCL24 monoclonal antibody. Lung function may be evaluated by determining any one of numerous measurements, such as expiratory reserve volume (ERV), forced vital capacity (FVC), forced expiratory volume (LEV) (e.g., LEV in one second, FEV1), FEV1/FEV ratio, forced expiratory flow 25% to 75%, and maximum voluntary ventilation (MVVpeak expiratory flow (PEF), slow vital capacity (SVC). Total lung volumes include total lung capacity (TEC), vital capacity (VC),), residual volume (RV), and functional residual capacity (FRC). In some embodiments, the subject suffers from systemic sclerosis (SSc). In some embodiments, the anti-CCL24 monoclonal antibody is CM-101.

### Nonalcoholic Steatohepatitis (NASH) Treatment

In some embodiments, the present disclosure provides methods of treating nonalcoholic steatohepatitis (NASH) in a human subject, comprising administering an effective amount of an anti-CCL24 monoclonal antibody or a pharmaceutical composition thereof. In some embodiments, the anti-CCL24 monoclonal antibody is CM-101. In some embodiments, the use of anti-CCL24 monoclonal antibody for treating nonalcoholic steatohepatitis (NASH) causes one or more effects in a human subject as described below.

In some embodiments, administration of the anti-CCL24 monoclonal antibody or a pharmaceutical composition thereof increases total sera level of CCL24 compared to baseline total sera level of CCL24 before administering the anti-CCL24 monoclonal antibody. In some embodiments, administration of the anti-CCL24 monoclonal antibody or a pharmaceutical composition thereof increases total sera level of CCL24 to at least 125%, at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured at about 8 hours, about 16 hours or about 24 hours post administration of the anti-CCL24 monoclonal antibody. In some embodiments, the increase of total sera level of CCL24 is measured at day 1, day 2, day 3, day 4, day 5, day 6, day 7, day 8, day 9, day 10 or day 11 post administration. In some embodiments, subcutaneous administration of 5 mg/kg of the anti-CCL24 monoclonal antibody increases total sera level of CCL24 at day 4 post administration to at least 150%, at least 175%, at least 200%, at least 250% or at least 300% of baseline total sera level of CCL24 before administering the anti-CCL24 monoclonal antibody. In some embodiments, intravenous administration of 10 mg/kg of the anti-CCL24 monoclonal antibody increases total sera level of CCL24 at day 4 post administration to at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured between the first and the second administrations of the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof improves hepatic condition of the subject as evaluated by one or more circulating fibrosis markers. In some embodiments, administration of the anti-CCL24 monoclonal antibody or a pharmaceutical composition thereof lowers the level of one or more fibrosis or inflammatory markers compared to baseline before administering the anti-CCL24 monoclonal antibody. Fibrosis or inflammatory markers include plasma alanine aminotransferase (ALT), alkaline phosphatase (ALP), aspartate aminotransferase (AST), PRO-C3, PRO-C4, PRO-05, C3M, cytokeratin 18 (CK18), amino-terminal propeptide of type III procollagen (PIIINP), hyaluronic acid (HA), Tissue Inhibitor of Metalloproteinases 1 (TIMP-1), Tissue Inhibitor of Metalloproteinases 2 (TIMP-2), platelet derived growth factor (PDGF), e.g. PDGF AA, γ-glutamyltransferase (GGT), bilirubin, C-reactive protein (CRP), erythrocyte sedimentation rate (ESR), and procalcitonin (PCT). Levels in the markers can be tested by well-established methods, for example, ELISA assay, or methods described in the examples herein.

In some embodiments, administration of the anti-CCL24 monoclonal antibody or a pharmaceutical composition thereof improves hepatic condition as evaluated by one or more imaging tests. In some embodiments, the imaging test is liver elastography. In some embodiments, the imaging test is an ultrasound elastography such as FibroScan. In some embodiments, the imaging test is magnetic resonance imaging. In some embodiments, the imaging test is Magnetic Resonance Imaging Proton Density Fat Fraction (MRI-PDFF). In some embodiments, the hepatic condition is liver fat content. In some embodiments, liver fat content is measured by MRI-PDFF, FibroScan, or other liver elastography and/or imaging tests.

In some embodiments, administration of the anti-CCL24 monoclonal antibody or a pharmaceutical composition thereof lowers the level of liver fat content compared to baseline before administering the anti-CCL24 monoclonal antibody. In some embodiments, administration of the anti-CCL24 monoclonal antibody or a pharmaceutical composition thereof lowers the level of liver fat content by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of liver fat content by about 5% to about 20%, about 10% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101. In some embodiments, the liver fat content is evaluated by Magnetic Resonance Imaging such as MRI-PDFF. In some embodiments, the liver fat content is measured by FibroScan CAP score.

FibroScan can provide fibrosis result which is a measurement of the amount of scarring in the liver. FibroScan measures scarring by measuring the stiffness of the liver. The fibrosis result is measured in kilopascals (kPa). In some embodiments, a range of 2 to 7 kPa indicates no liver scarring or mild liver scarring, a range of 7.5 to 10 kPa indicates moderate liver scarring, a range of 10 to 14 kPa indicates severe liver scarring, and a range of 14 kPa or higher indicates advanced liver scarring (cirrhosis). FibroScan can also provide CAP score which is a measurement of fatty change or fat content in the liver. The CAP score is measured in decibels per meter (dB/m). Typically, it ranges from 100 to 400 dB/m.

In some embodiments, administration of the anti-CCL24 monoclonal antibody or a pharmaceutical composition thereof decreases liver stiffness compared to baseline before administering the anti-CCL24 monoclonal antibody. In some embodiments, the liver stiffness is measured by FibroScan. In some embodiments, administration of the anti-CCL24 monoclonal antibody or a pharmaceutical composition thereof decreases liver stiffness, as measured by FibroScan fibrosis result, by at least 0.1 kPa, at least 0.2 kPa, at least 0.3 kPa, at least 0.5 kPa, at least 0.7 kPa, at least 1.0 kPa, at least 1.2 kPa, at least 1.5 kPa, at least 2.0 kPa, at least 2.5 kPa, at least 3.0 kPa, at least 3.5 kPa, at least 4.0 kPa, at least 5.0 kPa, at least 6.0 kPa, at least 8.0 kPa, at least 10 kPa, at least 15 kPa, at least 20 kPa, at least 25 kPa, or at least 30 kPa, compared to baseline before administering the anti-CCL24 monoclonal antibody. In some embodiments, administration of the anti-CCL24 monoclonal antibody or a pharmaceutical composition thereof decreases liver stiffness, as measured by FibroScan fibrosis result, by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases CAP score, as measured by FibroScan, by at least 5 dB/m, at least 10 dB/m, at least 15 dB/m, at least 20 dB/m, at least 30 dB/m, at least 40 dB/m, at least 50 dB/m, at least 60 dB/m, at least 80 dB/m, at least 100 dB/m, at least 120 dB/m, at least 150 dB/m, or at least 200 dB/m compared to baseline before administering the anti-CCL24 monoclonal antibody. In some embodiments, administration of the anti-CCL24 monoclonal antibody or a pharmaceutical composition thereof decreases CAP score, as measured by FibroScan, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases NAFLD activity score (NAS score), as measured by liver biopsy, by at least 1 point, at least 2 points, at least 3 points, at least 4 points, at least 5 points, at least 6 points, at least 7 points compared to baseline before administering the anti-CCL24 monoclonal antibody. In some embodiments, administration of the anti-CCL24 monoclonal antibody or a pharmaceutical composition thereof decreases NAS score, as measured by liver biopsy, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases fibrosis score, as measured by liver biopsy, by at least 1 point, at least 2 points, at least 3 points, at least 4 points compared to baseline before administering the anti-CCL24 monoclonal antibody. In some embodiments, administration of the anti-CCL24 monoclonal antibody or a pharmaceutical composition thereof decreases fibrosis score, as measured by liver biopsy, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

### Primary Sclerosing Cholangitis (PSC) Treatment

In some embodiments, the present disclosure provides methods of treating primary sclerosing cholangitis (PSC) in a human subject, comprising administering an effective amount of an anti-CCL24 monoclonal antibody or a pharmaceutical composition thereof. In some embodiments, the anti-CCL24 monoclonal antibody is CM-101. In some embodiments, the use of anti-CCL24 monoclonal antibody for treating primary sclerosing cholangitis (PSC) causes one or more effects in a human subject as described below.

In some embodiments, administration of the anti-CCL24 monoclonal antibody or a pharmaceutical composition thereof increases total sera level of CCL24 compared to baseline total sera level of CCL24 before administering the anti-CCL24 monoclonal antibody. In some embodiments, administration of the anti-CCL24 monoclonal antibody or a pharmaceutical composition thereof increases total sera level of CCL24 to at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured at about 8 hour, about 16 hour or about 24 hour post administration of the anti-CCL24 monoclonal antibody. In some embodiments, the increase of total sera level of CCL24 is measured at day 1, day 2, day 3, day 4, day 5, day 6, day 7, day 8, day 9, day 10 or day 11 post administration. In some embodiments, subcutaneous administration of 5 mg/kg of the anti-CCL24 monoclonal antibody increases total sera level of CCL24 at day 4 post administration to at least 150%, at least 175%, at least 200%, at least 250% or at least 300% of baseline total sera level of CCL24 before administering the anti-CCL24 monoclonal antibody. In some embodiments, intravenous administration of 10 mg/kg of the anti-CCL24 monoclonal antibody increases total sera level of CCL24 at day 4 post administration to at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured between the first and the second administrations of the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof improves hepatic condition of the subject as evaluated by one or more circulating fibrosis markers. In some embodiments, administration of the anti-CCL24 monoclonal antibody or a pharmaceutical composition thereof lowers the level of one or more fibrosis or inflammatory markers compared to baseline before administering the anti-CCL24 monoclonal antibody. Fibrosis or inflammatory markers include plasma alanine aminotransferase (ALT), alkaline phosphatase (ALP), aspartate aminotransferase (AST), PRO-C3, PRO-C4, PRO-05, C3M, cytokeratin 18 (CK18), amino-terminal propeptide of type III procollagen (PIIINP), hyaluronic acid (HA), Tissue Inhibitor of Metalloproteinases 1 (TIMP-1), Tissue Inhibitor of Metalloproteinases 2 (TIMP-2), platelet derived growth factor (PDGF), e.g. PDGF AA, γ-glutamyltransferase (GGT), bilirubin, C-reactive protein (CRP), erythrocyte sedimentation rate (ESR), and procalcitonin (PCT). Levels in the markers can be tested by well-established methods, for example, ELISA assay, or methods described in the examples herein.

In some embodiments, administration of the anti-CCL24 monoclonal antibody or a pharmaceutical composition thereof lowers the level of circulating alkaline phosphatase (ALP) compared to baseline before administering the anti-CCL24 monoclonal antibody. In some embodiments, administration of the anti-CCL24 monoclonal antibody or a pharmaceutical composition thereof lowers the level of circulating alkaline phosphatase (ALP) by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of circulating alkaline phosphatase (ALP) by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers Enhanced Liver Fibrosis (ELF) score compared to baseline before administering the anti-CCL24 monoclonal antibody. In some embodiments, administration of the anti-CCL24 monoclonal antibody or a pharmaceutical composition thereof lowers Enhanced Liver Fibrosis (ELF) score by at least about 0.1, at least about 0.2, at least about 0.3, at least about 0.5, at least about 0.7, at least about 1.0, at least about 1.5, at least about 2, at least about 3, at least about 4, at least about 5, or more than 5, compared to baseline before administering the anti-CCL24 monoclonal antibody. The Enhanced Liver Fibrosis (ELF) test is a non-invasive blood test that measures three direct markers of fibrosis: hyaluronic acid (HA), procollagen III amino-terminal peptide (PIIINP), and tissue inhibitor of matrix metalloproteinase 1 (TIMP-1). The ELF test, in conjunction with other laboratory and clinical findings, can be used to assess the risk of progression to cirrhosis and LREs in patients with chronic liver disease. In some embodiments, an ELF score of <7.7 indicates no to mild fibrosis, an ELF score of 7.7 to 9.8 indicates moderate fibrosis, an ELF score of 9.8 to 11.3 indicates severe fibrosis, and an ELF score of >=11.3 indicates cirrhosis.

In some embodiments, administration of the anti-CCL24 monoclonal antibody or a pharmaceutical composition thereof lowers Enhanced Liver Fibrosis (ELF) score by at least 0.1, at least 0.2, at least 0.3, at least 0.5, at least 0.7, at least 0.8, at least 1.0, at least 1.2, at least 1.5, at least 2.0, at least 2.5, at least 3.0, at least 3.5, at least 4.0, at least 5.0, at least 6.0, at least 7.0, at least 8.0, or at least 9.0 compared to baseline before administering the anti-CCL24 monoclonal antibody. In some embodiments, administration of the anti-CCL24 monoclonal antibody or a pharmaceutical composition thereof lowers Enhanced Liver Fibrosis (ELF) score by about 0.1 to about 1, about 0.5 to about 1.5, about 1.0 to about 3.0, about 1.5 to about 4.0, or about 2.0 to about 6.0 compared to baseline before administering the anti-CCL24 monoclonal antibody. In some embodiments, administration of the anti-CCL24 monoclonal antibody or a pharmaceutical composition thereof lowers Enhanced Liver Fibrosis (ELF) score to less than 11.5, less than 11.0, less than 10.5, less than 10.0, less than 9.5, less than 9.0, less than 8.5, less than 8.0, less than 7.5, or less than 7.0. In some embodiments, the anti-CCL24 monoclonal antibody is CM-101.

In some embodiments, administration of the anti-CCL24 monoclonal antibody or a pharmaceutical composition thereof lowers the level of circulating PRO-C3 (pro-peptide of type III collagen) compared to baseline before administering the anti-CCL24 monoclonal antibody. In some embodiments, administration of the anti-CCL24 monoclonal antibody or a pharmaceutical composition thereof lowers the level of PRO-C3 by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of PRO-C3 by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating PRO-C4 compared to baseline before administering the anti-CCL24 monoclonal antibody. In some embodiments, administration of the anti-CCL24 monoclonal antibody or a pharmaceutical composition thereof lowers the level of PRO-C4 by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of PRO-C4 by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating C3M compared to baseline before administering the anti-CCL24 monoclonal antibody. In some embodiments, administration of the anti-CCL24 monoclonal antibody or a pharmaceutical composition thereof lowers the level of C3M by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of C3M by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof lowers the level of circulating PRO-05 (pro-peptide of type V collagen) compared to baseline before administering the anti-CCL24 monoclonal antibody. In some embodiments, administration of the anti-CCL24 monoclonal antibody or a pharmaceutical composition thereof lowers the level of PRO-05 by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, administration of the anti-CCL24 monoclonal antibody or a pharmaceutical composition thereof lowers the level of PRO-05 by about 5% to about 30%, about 20% to about 50%, about 40% to about 70%, or about 60% to about 99% compared to baseline before administering the anti-CCL24 monoclonal antibody. In some embodiments, the anti-CCL24 monoclonal antibody is CM-101.

FibroScan can provide fibrosis result which is a measurement of the amount of scarring in the liver. FibroScan measures scarring by measuring the stiffness of the liver. The fibrosis result is measured in kilopascals (kPa). In some embodiments, a range of 2 to 7 kPa indicates no liver scarring or mild liver scarring, a range of 7.5 to 10 kPa indicates moderate liver scarring, a range of 10 to 14 kPa indicates severe liver scarring, and a range of 14 kPa or higher indicates advanced liver scarring (cirrhosis). FibroScan can also provide CAP score which is a measurement of fatty change or fat content in the liver. The CAP score is measured in decibels per meter (dB/m). Typically, it ranges from 100 to 400 dB/m.

In some embodiments, administration of the anti-CCL24 monoclonal antibody or a pharmaceutical composition thereof decreases liver stiffness compared to baseline before administering the anti-CCL24 monoclonal antibody. In some embodiments, the liver stiffness is measured by FibroScan. In some embodiments, administration of the anti-CCL24 monoclonal antibody or a pharmaceutical composition thereof decreases liver stiffness, as measured by FibroScan fibrosis result, by at least 0.1 kPa, at least 0.2 kPa, at least 0.3 kPa, at least 0.5 kPa, at least 0.7 kPa, at least 1.0 kPa, at least 1.2 kPa, at least 1.5 kPa, at least 2.0 kPa, at least 2.5 kPa, at least 3.0 kPa, at least 3.5 kPa, at least 4.0 kPa, at least 5.0 kPa, at least 6.0 kPa, at least 8.0 kPa, at least 10 kPa, at least 15 kPa, at least 20 kPa, at least 25 kPa, or at least 30 kPa, compared to baseline before administering the anti-CCL24 monoclonal antibody. In some embodiments, administration of the anti-CCL24 monoclonal antibody or a pharmaceutical composition thereof decreases liver stiffness, as measured by FibroScan fibrosis result, by at least about 5%, at least about 10%, at least about 15%, at least about 20%, at least about 25%, at least about 30%, at least about 40%, at least about 50%, at least about 60%, at least about 70%, at least about 80%, at least about 90%, or more than 90% compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases CAP score, as measured by FibroScan, by at least 5 dB/m, at least 10 dB/m, at least 15 dB/m, at least 20 dB/m, at least 30 dB/m, at least 40 dB/m, at least 50 dB/m, at least 60 dB/m, at least 80 dB/m, at least 100 dB/m, at least 120 dB/m, at least 150 dB/m, or at least 200 dB/m compared to baseline before administering the anti-CCL24 monoclonal antibody. In some embodiments, administration of the anti-CCL24 monoclonal antibody or a pharmaceutical composition thereof decreases CAP score, as measured by FibroScan, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof decreases histological damage, as measured by liver biopsy, compared to baseline before administering the anti-CCL24 monoclonal antibody. In some embodiments, administration of the anti-CCL24 monoclonal antibody or a pharmaceutical composition thereof decreases histological liver damage, as measured by liver biopsy, by at least about 5%, at least about 10%, at least about 15%, at least about 20%, at least about 25%, at least about 30%, at least about 40%, at least about 50%, at least about 60%, or at least about 70%, compared to baseline before administering the anti-CCL24 monoclonal antibody. In some embodiments, the anti-CCL24 monoclonal antibody is CM-101.

### Systemic Sclerosis (SSc) Treatment

In some embodiments, the present disclosure provides methods of treating systemic sclerosis (SSc) in a human subject, comprising administering an effective amount of an anti-CCL24 monoclonal antibody or a pharmaceutical composition thereof. In some embodiments, the anti-CCL24 monoclonal antibody is CM-101. In some embodiments, the use of anti-CCL24 monoclonal antibody for treating systemic sclerosis (SSc) causes one or more effects in a human subject as described below.

In some embodiments, administration of the anti-CCL24 monoclonal antibody or a pharmaceutical composition thereof improves Combined Response Index for Systemic Sclerosis (CRISS) score of the subject. The calculation of CRISS is a 2-step process for use in a clinical trial and is described in Khanna et. al., Arthritis Rheumatol. 2016 February; 68(2):299-311. In some embodiments, subjects for whom the predicted probability is >=0.60 are considered improved, while subjects for whom the predicted probability is <0.60 are considered not improved. In some embodiments, the administration of the anti-CCL24 monoclonal antibody or a pharmaceutical composition thereof results in an average CRISS score of about 0.1, about 0.2, about 0.1, about 0.3, about 0.4, about 0.5, about 0.6, about 0.7, about 0.8, about 0.9, or about 1.0. In some embodiments, the administration of the anti-CCL24 monoclonal antibody or a pharmaceutical composition thereof results in an average CRISS score of at least 0.1, at least 0.2, at least 0.1, at least 0.3, at least 0.4, at least 0.5, at least 0.6, at least 0.7, at least 0.8, at least 0.9, or at least 1.0. In some embodiments, administration of the anti-CCL24 monoclonal antibody or a pharmaceutical composition improves lung function of the subject compared to baseline before administering the anti-CCL24 monoclonal antibody in subjects suffering from Systemic Sclerosis (SSc). In some embodiments, the anti-CCL24 monoclonal antibody is CM-101.

In some embodiments, administration of the anti-CCL24 monoclonal antibody or a pharmaceutical composition thereof improves dermal thickness of the subject compared to baseline before administering the anti-CCL24 monoclonal antibody. In some embodiments, administration of the anti-CCL24 monoclonal antibody or a pharmaceutical composition thereof improves dermal thickness to be evaluated using modified Rodnan Score (mRss) of the subject by at least about 5%, at least about 10%, at least about 20%, at least about 30%, at least about 40%, at least about 50%, at least about 60%, at least about 70%, at least about 80%, at least about 90%, at least about 100%, compared to baseline before administering the anti-CCL24 monoclonal antibody.

In some embodiments, administration of the anti-CCL24 monoclonal antibody or a pharmaceutical composition thereof improves lung function of the subject compared to baseline before administering the anti-CCL24 monoclonal antibody. In some embodiments, administration of the anti-CCL24 monoclonal antibody or a pharmaceutical composition thereof improves lung function of the subject by at least about 5%, at least about 10%, at least about 20%, at least about 30%, at least about 40%, at least about 50%, at least about 60%, at least about 70%, at least about 80%, at least about 90%, at least about 100%, at least about 150% or at least about 200%, compared to baseline before administering the anti-CCL24 monoclonal antibody. Lung function may be evaluated by determining any one of numerous measurements, such as expiratory reserve volume (ERV), forced vital capacity (FVC), forced expiratory volume (LEV) (e.g., LEV in one second, FEV1), FEV1/FEV ratio, forced expiratory flow 25% to 75%, and maximum voluntary ventilation (MVVpeak expiratory flow (PEF), slow vital capacity (SVC). Total lung volumes include total lung capacity (TEC), vital capacity (VC),), residual volume (RV), and functional residual capacity (FRC). In some embodiments, the anti-CCL24 monoclonal antibody is CM-101.

In some embodiments, administration of the anti-CCL24 monoclonal antibody or a pharmaceutical composition thereof increases total sera level of CCL24 compared to baseline total sera level of CCL24 before administering the anti-CCL24 monoclonal antibody. In some embodiments, administration of the anti-CCL24 monoclonal antibody or a pharmaceutical composition thereof increases total sera level of CCL24 to at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured at about 8 hour, about 16 hour or about 24 hour post administration of the anti-CCL24 monoclonal antibody. In some embodiments, the increase of total sera level of CCL24 is measured at day 1, day 2, day 3, day 4, day 5, day 6, day 7, day 8, day 9, day 10 or day 11 post administration. In some embodiments, subcutaneous administration of 5 mg/kg of the anti-CCL24 monoclonal antibody increases total sera level of CCL24 at day 4 post administration to at least 150%, at least 175%, at least 200%, at least 250% or at least 300% of baseline total sera level of CCL24 before administering the anti-CCL24 monoclonal antibody. In some embodiments, intravenous administration of 10 mg/kg of the anti-CCL24 monoclonal antibody increases total sera level of CCL24 at day 4 post administration to at least 150%, at least 200%, at least 250%, at least 300%, at least 350%, at least 400%, at least 450%, or at least 500% of baseline total sera level of CCL24 before administering the anti-CCL24 monoclonal antibody. In some embodiments, the increase of total sera level of CCL24 is measured between the first and the second administrations of the anti-CCL24 monoclonal antibody. In some embodiments, the anti-CCL24 monoclonal antibody is CM-101.

In some embodiments, administration of the anti-CCL24 monoclonal antibody or a pharmaceutical composition thereof improves hepatic condition of the subject as evaluated by one or more circulating fibrosis markers. In some embodiments, administration of the anti-CCL24 monoclonal antibody or a pharmaceutical composition thereof lowers the level of one or more fibrosis or inflammatory markers compared to baseline before administering the anti-CCL24 monoclonal antibody. PRO-C3, PRO-C4, PRO-05, C3M, cytokeratin 18 (CK18), amino-terminal propeptide of type III procollagen (PIIINP), hyaluronic acid (HA), Tissue Inhibitor of Metalloproteinases 1 (TIMP-1), Tissue Inhibitor of Metalloproteinases 2 (TIMP-2), platelet derived growth factor (PDGF), e.g., PDGF AA, γ-glutamyltransferase (GGT), bilirubin, C-reactive protein (CRP), erythrocyte sedimentation rate (ESR), and procalcitonin (PCT). Levels in the markers can be tested by well-established methods, for example, ELISA assay, or methods described in the examples herein.

### Safety

In some embodiments, administration of the anti-CCL24 monoclonal antibody or a pharmaceutical composition thereof according to the one or more doses and/or one or more schedules as described herein causes no dose limiting toxicity. In some embodiments, administration of the anti-CCL24 monoclonal antibody or a pharmaceutical composition thereof causes no injection site reaction. In some embodiments, administration of the anti-CCL24 monoclonal antibody or a pharmaceutical composition thereof causes no severe or serious adverse event (AE). In some embodiments, the anti-CCL24 monoclonal antibody is CM-101.

In some embodiments, administration of the anti-CCL24 monoclonal antibody or a pharmaceutical composition thereof in a subject does not elevate level of triglycerides (TG), low-density lipoprotein (LDL) and/or Cholesterol in the subject's blood. In some embodiments, administration of the anti-CCL24 monoclonal antibody or a pharmaceutical composition thereof in a subject reduces the level of triglycerides (TG), low-density lipoprotein (LDL) and/or Cholesterol in the subject's blood by less than 5%, less than 10%, less than 15%, less than 20%, less than 30%, less than 40%, less than 50%, less than 75%, or less than 100% compared to the baseline before administering the anti-CCL24 monoclonal antibody. In some embodiments, the anti-CCL24 monoclonal antibody is CM-101.

### Administration Dose

In some embodiments, the anti-CCL24 monoclonal antibody (e.g., CM-101) is administered at a given dose. In some embodiments, the dose is between about 0.1 mg/kg to about 100 mg/kg. In some embodiments, the dose is above 100 mg/kg. In some embodiments, the dose is between about 1 mg/kg to about 100 mg/kg. In some embodiments, the dose is between about 1 mg/kg to about 20 mg/kg. In some embodiments, the dose is between about 10 mg/kg to about 30 mg/kg. In some embodiments, the dose is between about 20 mg/kg to about 40 mg/kg. In some embodiments, the dose is between about 30 mg/kg to about 50 mg/kg. In some embodiments, the dose is between about 40 mg/kg to about 60 mg/kg. In some embodiments, the dose is between about 50 mg/kg to about 70 mg/kg. In some embodiments, the dose is between about 60 mg/kg to about 80 mg/kg. In some embodiments, the dose is between about 70 mg/kg to about 90 mg/kg. In some embodiments, the dose is between about 80 mg/kg to about 100 mg/kg. In some embodiments, the dose is between about 0.1 mg/kg to about 1 mg/kg. In some embodiments, the dose is between about 0.5 mg/kg to about 3 mg/kg. In some embodiments, the dose is between about 1 mg/kg to about 6 mg/kg. In some embodiments, the dose is between about 3 mg/kg to about 8 mg/kg. In some embodiments, the dose is between about 5 mg/kg to about 10 mg/kg. In some embodiments, the dose is between about 7 mg/kg to about 12 mg/kg. In some embodiments, the dose is between about 9 mg/kg to about 14 mg/kg. In some embodiments, the dose is between about 11 mg/kg to about 16 mg/kg. In some embodiments, the dose is between about 13 mg/kg to about 18 mg/kg. In some embodiments, the dose is between about 15 mg/kg to about 20 mg/kg. In some embodiments, the dose is between about 15 mg/kg to about 20 mg/kg.

In some embodiments, the dose is about 0.1 mg/kg, about 0.25 mg/kg, about 0.5 mg/kg, about 0.75 mg/kg, about 1 mg/kg, about 1.5 mg/kg, or about 2 mg/kg. In some embodiments, the dose is about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the dose is about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In some embodiments, the dose is about 30 mg/kg, about 40 mg/kg, about 50 mg/kg, about 60 mg/kg, about 70 mg/kg, about 80 mg/kg, about 90 mg/kg, or about 100 mg/kg.

In some embodiments, the anti-CCL24 monoclonal antibody (e.g., CM-101) is administered intravenously at a given dose. In some embodiments, the dose is about 0.75 mg/kg, about 2.5 mg/kg, about 5 mg/kg, about 10 mg/kg, about 15 mg/kg, or about 20 mg/kg. In some embodiments, the dose is about 0.75 mg/kg. In some embodiments, the dose is about 2.5 mg/kg. In some embodiments, the dose is about 5 mg/kg. In some embodiments, the dose is about 10 mg/kg. In some embodiments, the dose is about 15 mg/kg. In some embodiments, the dose is about 20 mg/kg. In some embodiments, intravenous administration of the anti-CCL24 monoclonal antibody (e.g., CM-101) is for treating primary sclerosing cholangitis (PSC)

In some embodiments, the anti-CCL24 monoclonal antibody (e.g., CM-101) is administered subcutaneously at a given dose. In some embodiments, the dose is about 0.75 mg/kg, about 2.5 mg/kg, about 5 mg/kg, or about 10 mg/kg. In some embodiments, the dose is about 0.75 mg/kg. In some embodiments, the dose is about 2.5 mg/kg. In some embodiments, the dose is about 5 mg/kg. In some embodiments, the dose is about 10 mg/kg. In some embodiments, subcutaneous administration of the anti-CCL24 monoclonal antibody (e.g., CM-101) is for treating nonalcoholic steatohepatitis (NASH).

### Administration Schedule

Administration according to the present disclosure may be performed by any of the following routes: oral administration, intravenous, intramuscular, intraperitoneal, intratechal or subcutaneous injection; intrarectal administration; intranasal administration, ocular administration or topical administration.

In some embodiments, administration according to the present disclosure is performed intravenously. In some embodiments, administration according to the present disclosure is performed subcutaneously.

In some embodiments, the anti-CCL24 monoclonal antibody (e.g., CM-101) is administered once every one to ten weeks. In some embodiments, the anti-CCL24 monoclonal antibody is administered once every one to five weeks. In some embodiments, the anti-CCL24 monoclonal antibody is administered once every one to four weeks. In some embodiments, the anti-CCL24 monoclonal antibody is administered once every one to three weeks. In some embodiments, the anti-CCL24 monoclonal antibody is administered once every one to two weeks. In some embodiments, the anti-CCL24 monoclonal antibody is administered once every two to ten weeks. In some embodiments, the anti-CCL24 monoclonal antibody is administered once every two to five weeks. In some embodiments, the anti-CCL24 monoclonal antibody is administered once every two to four weeks. In some embodiments, the anti-CCL24 monoclonal antibody is administered once every two to three weeks. In some embodiments, the anti-CCL24 monoclonal antibody is administered once every three to ten weeks. In some embodiments, the anti-CCL24 monoclonal antibody is administered once every three to five weeks. In some embodiments, the anti-CCL24 monoclonal antibody is administered once every three to four weeks. In some embodiments, the anti-CCL24 monoclonal antibody is administered once every four to ten weeks. In some embodiments, the anti-CCL24 monoclonal antibody is administered once every four to five weeks. In some embodiments, the anti-CCL24 monoclonal antibody is administered once every about 1, about 2, about 3, about 4, about 5, about 6, about 7, about 8, about 9, about 10 weeks, or more than 10 weeks. In some embodiments, the anti-CCL24 monoclonal antibody is administered once every about 1, about 2, about 3, about 4, about 5, about 6, about 7, about 8, about 9, about 10, about 11, about 12, or about 13 days.

In some embodiments, the anti-CCL24 monoclonal antibody (e.g., CM-101) is administered intravenously according to a given administration frequency. In some embodiments, the anti-CCL24 monoclonal antibody is administered once every one to two weeks. In some embodiments, the anti-CCL24 monoclonal antibody is administered once every one to three weeks. In some embodiments, the anti-CCL24 monoclonal antibody is administered once every two to three weeks. In some embodiments, the anti-CCL24 monoclonal antibody is administered once every two to four weeks. In some embodiments, the anti-CCL24 monoclonal antibody is administered once every three to four weeks. In some embodiments, the anti-CCL24 monoclonal antibody is administered once every three to five weeks. In some embodiments, the anti-CCL24 monoclonal antibody is administered once every about 1, about 2, about 3, about 4, about 5, about 6, about 7, about 8, about 9, or about 10 weeks. In some embodiments, the anti-CCL24 monoclonal antibody is administered once every, about 7, about 8, about 9, about 10, about 11, about 12, or about 13 days. In some embodiments, the anti-CCL24 monoclonal antibody is administered once every about two weeks. In some embodiments, the anti-CCL24 monoclonal antibody is administered once every about three weeks. In some embodiments, the anti-CCL24 monoclonal antibody is administered once every about four weeks.

In some embodiments, the anti-CCL24 monoclonal antibody (e.g., CM-101) is administered subcutaneously according to a given administration frequency. In some embodiments, the anti-CCL24 monoclonal antibody is administered once every one to two weeks. In some embodiments, the anti-CCL24 monoclonal antibody is administered once every one to three weeks. In some embodiments, the anti-CCL24 monoclonal antibody is administered once every two to three weeks. In some embodiments, the anti-CCL24 monoclonal antibody is administered once every two to four weeks. In some embodiments, the anti-CCL24 monoclonal antibody is administered once every three to four weeks. In some embodiments, the anti-CCL24 monoclonal antibody is administered once every three to five weeks. In some embodiments, the anti-CCL24 monoclonal antibody is administered once every about 1, about 2, about 3, about 4, about 5, about 6, about 7, about 8, about 9, or about 10 weeks. In some embodiments, the anti-CCL24 monoclonal antibody is administered once every about 1, about 2, about 3, about 4, about 5, about 6, about 7, about 8, about 9, about 10, about 11, about 12, or about 13 days. In some embodiments, the anti-CCL24 monoclonal antibody is administered once every about two weeks. In some embodiments, the anti-CCL24 monoclonal antibody is administered once every about three weeks. In some embodiments, the anti-CCL24 monoclonal antibody is administered once every about four weeks.

In some embodiments, the anti-CCL24 monoclonal antibody (e.g., CM-101) is administered according to the administration frequency described herein for a duration of time. In some embodiments, the anti-CCL24 monoclonal antibody is administered for at least 4, at least 6, at least 8, at least 10, at least 12, at least 14, at least 15, at least 16, at least 18, at least 20, or at least 24 weeks. In some embodiments, the anti-CCL24 monoclonal antibody is administered for at least 1, at least 2, at least 4, at least 6, at least 9, or at least 12 months. In some embodiments, the anti-CCL24 monoclonal antibody is administered for about 4 to about 8, about 6 to about 12, about 8 to about 16, about 10 to about 20, about 15 to about 30, or about 20 to about 40 weeks.

### Pharmacokinetics of CM-101

In some embodiments, CM-101 is administered according to an administration schedule described herein to maintain a steady-state serum concentration of CM-101 in a subject. In some embodiments, the subject maintains a steady-state serum concentration of CM-101 of about 1 to about 5000 μg/mL. In some embodiments, the subject maintains a steady-state serum concentration of CM-101 of about 10 to about 2000 μg/mL. In some embodiments, the subject maintains a steady-state serum concentration of CM-101 of about 1 to about 300 μg/mL. In some embodiments, the subject maintains a steady-state serum concentration of CM-101 of about 100 to about 500 μg/mL. In some embodiments, the subject maintains a steady-state serum concentration of CM-101 of about 200 to about 800 μg/mL. In some embodiments, the subject maintains a steady-state serum concentration of CM-101 of about 300 to about 1000 μg/mL. In some embodiments, the subject maintains a steady-state serum concentration of CM-101 of about 400 to about 2000 μg/mL. In some embodiments, the subject maintains a steady-state serum concentration of CM-101 of about 500 to about 5000 μg/mL.

In some embodiments, the subject maintains a steady-state serum concentration of CM-101 of about 10 to about 500 μg/mL. In some embodiments, the subject maintains a steady-state serum concentration of CM-101 of about 20 to about 400 μg/mL. In some embodiments, the subject maintains a steady-state serum concentration of CM-101 of about 30 to about 300 μg/mL. In some embodiments, the subject maintains a steady-state serum concentration of CM-101 of about 40 to about 200 μg/mL. In some embodiments, the subject maintains a steady-state serum concentration of CM-101 of about 50 to about 100 μg/mL. In some embodiments, the subject maintains a steady-state serum concentration of CM-101 of about 60 to about 90 μg/mL.

In some embodiments, the subject maintains a steady-state serum concentration of CM-101 of about 10 to about 1000 μg/mL. In some embodiments, the subject maintains a steady-state serum concentration of CM-101 of about 20 to about 800 μg/mL. In some embodiments, the subject maintains a steady-state serum concentration of CM-101 of about 40 to about 600 μg/mL. In some embodiments, the subject maintains a steady-state serum concentration of CM-101 of about 50 to about 500 μg/mL. In some embodiments, the subject maintains a steady-state serum concentration of CM-101 of about 60 to about 400 μg/mL. In some embodiments, the subject maintains a steady-state serum concentration of CM-101 of about 70 to about 300 μg/mL.

In some embodiments, CM-101 reaches the steady-state serum concentration as described herein following about 1 to about 10 administrations. In some embodiments, CM-101 reaches the steady-state serum concentration as described herein following about 1 to about 4, about 2 to about 5, about 3 to about 6, about 4 to about 8, or about 5 to about 10 administrations. In some embodiments, CM-101 reaches the steady-state serum concentration as described herein following about 1 to about 3, about 2 to about 4, about 3 to about 5, about 4 to about 6, or about 5 to about 7 administrations. In some embodiments, CM-101 reaches the steady-state serum concentration as described herein following about 1 to about 2, about 2 to about 3, about 3 to about 4, about 4 to about 5, about 5 to about 6, or about 6 to about 7 administrations. In some embodiments, CM-101 reaches the steady-state serum concentration as described herein following about 1, about 2, about 3, about 4, about 5, about 6, about 7, or about 8 administrations. In some embodiments, CM-101 reaches the steady-state serum concentration as described herein following about two administrations. In some embodiments, CM-101 reaches the steady-state serum concentration as described herein following about three administrations. In some embodiments, CM-101 reaches the steady-state serum concentration as described herein following about four administrations.

In some embodiments, the half-life of CM-101 following the administration route, the administration dose, and/or the administration schedule as described herein is 5-40 days. In some embodiments, the half-life of CM-101 is about 10-30 days. In some embodiments, the half-life of CM-101 is about 5 to about 15, about 10 to about 20, about 15 to about 25, or about 20 to about 30 days. In some embodiments, the half-life of CM-101 is about 10 to about 15, about 15-about 20, about 20 to about 25, or about 25 to about 30 days. In some embodiments, the half-life of CM-101 is about 10, 11, 12, 13, 14, 15, 16, 17, 18, 19, 20, 21, 22, 23, 24, 25, 26, 27, 28, 29, or 30 days. In some embodiments, the half-life of CM-101 is about 15 days. In some embodiments, the half-life of CM-101 is about 16 days. In some embodiments, the half-life of CM-101 is about 17 days. In some embodiments, the half-life of CM-101 is about 18 days. In some embodiments, the half-life of CM-101 is about 19 days. In some embodiments, the half-life of CM-101 is about 20 days. In some embodiments, the half-life of CM-101 is about 21 days.

### Pharmaceutical Composition and Kit

In one aspect, the present disclosure provides a pharmaceutical composition comprising as an active ingredient the anti-CCL24 monoclonal antibody as described herein and a pharmaceutically acceptable carrier, excipient or diluent. In some embodiments, the pharmaceutical composition comprises the anti-CCL24 monoclonal antibody and a buffering agent, an agent which adjusts the osmolarity of the composition and optionally, one or more pharmaceutically acceptable carriers, excipients and/or diluents. In some embodiments, the anti-CCL24 monoclonal antibody is CM-101.

In some embodiments, the pharmaceutical composition according to the disclosure further comprises an additional therapeutic agent. Non-limiting examples of additional therapeutic agents include FXR agonists, PPAR agonists, anti-chemokine or anti-cytokine monoclonal antibodies, or any small molecule, protein or peptide that inhibit the activity of a chemokine or cytokine, anti-inflammatory agents including steroids and anti-fibrotic agents.

In some embodiments, the pharmaceutical composition of CM-101 comprises about 1 to about 500 mg/mL CM-101. In some embodiments, the pharmaceutical composition comprises about 100 to about 250 mg/mL CM-101. In some embodiments, the pharmaceutical composition of CM-101 comprises greater than 100 mg/mL, greater than 125 mg/mL, greater than 150 mg/mL, greater than 175 mg/mL, greater than 200 mg/mL, greater than 225 mg/mL or greater than 250 mg/mL CM-101. In some embodiments, the pharmaceutical composition of CM-101 comprises about 100 to about 150 mg/mL, about 150 to about 200 mg/mL or about 200 to about 250 mg/mL CM-101. In some embodiments, the pharmaceutical composition of CM-101 comprises about 100 mg/mL, about 125 mg/mL, about 150 mg/mL, about 175 mg/mL, about 200 mg/mL, about 225 mg/mL, or about 250 mg/mL CM-101. In some embodiments, the pharmaceutical composition of CM-101 comprises about 175 mg/mL CM-101.

In some embodiments, the pharmaceutical composition comprises one or more ingredients selected from the group consisting of histidine, phosphate, arginine, glutamic acid, sorbitol, polysorbate and sodium chloride.

In some embodiments, the pharmaceutical composition comprises histidine. In some embodiments, the pharmaceutical composition comprises histidine at about 1 to about 100 mM concentration. In some embodiments, the pharmaceutical composition comprises histidine at about 10 mM concentration. In some embodiments, the histidine is L-histidine.

In some embodiments, the pharmaceutical composition comprises phosphate. In some embodiments, the pharmaceutical composition comprises phosphate at about 1 to about 100 mM concentration. In some embodiments, the pharmaceutical composition comprises phosphate at about 10 mM concentration. In some embodiments, the phosphate is potassium phosphate.

In some embodiments, the pharmaceutical composition comprises arginine. In some embodiments, the pharmaceutical composition comprises arginine at about 30 to about 200 mM concentration. In some embodiments, the pharmaceutical composition comprises arginine at about 150 mM concentration.

In some embodiments, the pharmaceutical composition comprises glutamic acid. In some embodiments, the pharmaceutical composition comprises glutamic acid at about 30 to about 200 mM concentration. In some embodiments, the pharmaceutical composition comprises glutamic acid at about 150 mM concentration.

In some embodiments, the pharmaceutical composition comprises sorbitol. In some embodiments, the pharmaceutical composition comprises sorbitol at about 1% to about 5% concentration. In some embodiments, the pharmaceutical composition comprises sorbitol at about 3% concentration.

In some embodiments, the pharmaceutical composition comprises polysorbate. In some embodiments, the pharmaceutical composition comprises polysorbate at about 0.005% to about 0.05% concentration. In some embodiments, the pharmaceutical composition comprises polysorbate at about 0.02% concentration. In some embodiments, the polysorbate is polysorbate-80.

In some embodiments, the pharmaceutical composition comprises sodium chloride. In some embodiments, the pharmaceutical composition comprises sodium chloride at about 50 to about 150 mM concentration. In some embodiments, the pharmaceutical composition comprises sodium chloride at about 80 mM concentration.

In some embodiments, the pharmaceutical composition has a pH of about 5 to about 8. In some embodiments, the pharmaceutical composition has a pH of about 6 to about 7. In some embodiments, the pharmaceutical composition has a pH of about 5.5 to about 6.5. In some embodiments, the pharmaceutical composition has a pH of 5.5, 5.6, 5.7, 5.8, 5.9, 6.0, 6.1, 6.2, 6.3, 6.4, or 6.5. In some embodiments, the pharmaceutical composition has a pH of 6.1.

In some embodiments, the pharmaceutical composition comprises about 175 mg/mL CM-101, about 10 mM L-Histidine, about 150 mM Arginine, about 150 mM Glutamic Acid, about 0.02% (w/v) Polysorbate 80, and has a pH of about 6.1. In some embodiments, the pharmaceutical composition is for subcutaneous administration. In some embodiments, one vial of the pharmaceutical contains a fill volume of about 0.62 mL and/or a withdrawal volume of 0.5 mL. In some embodiments, the viscosity of the pharmaceutical composition is about 12.25 mPa*s at 20° C.

In some embodiments, the pharmaceutical composition of CM-101 comprises about 1 to about 150 mg/mL CM-101. In some embodiments, the pharmaceutical composition comprises about 5 to about 50 mg/mL CM-101. In some embodiments, the pharmaceutical composition of CM-101 comprises about 5 to about 20 mg/mL, about 20 to about 50 mg/mL or about 50 to about 100 mg/mL CM-101. In some embodiments, the pharmaceutical composition of CM-101 comprises about 1 mg/mL, about 2.5 mg/mL, about 5 mg/mL, about 7.5 mg/mL, about 10 mg/mL, about 15 mg/mL, about 20 mg/mL, about 30 mg/mL, or about 50 mg/mL CM-101. In some embodiments, the pharmaceutical composition of CM-101 comprises about 10 mg/mL CM-101.

In some embodiments, the pharmaceutical composition comprises histidine. In some embodiments, the pharmaceutical composition comprises histidine at about 1 to about 100 mM concentration. In some embodiments, the pharmaceutical composition comprises histidine at about 10 mM concentration. In some embodiments, the histidine is L-histidine.

In some embodiments, the pharmaceutical composition comprises phosphate. In some embodiments, the pharmaceutical composition comprises phosphate at about 1 to about 100 mM concentration. In some embodiments, the pharmaceutical composition comprises phosphate at about 10 mM concentration. In some embodiments, the phosphate is potassium phosphate.

In some embodiments, the pharmaceutical composition comprises glycine. In some embodiments, the pharmaceutical composition comprises glycine at about 10 to about 200 mM concentration. In some embodiments, the pharmaceutical composition comprises glycine at about 40 mM concentration.

In some embodiments, the pharmaceutical composition comprises sorbitol. In some embodiments, the pharmaceutical composition comprises sorbitol at about 1% to about 7% concentration. In some embodiments, the pharmaceutical composition comprises sorbitol at about 4% concentration.

In some embodiments, the pharmaceutical composition comprises polysorbate. In some embodiments, the pharmaceutical composition comprises polysorbate at about 0.005% to about 0.05% concentration. In some embodiments, the pharmaceutical composition comprises polysorbate at about 0.01% concentration. In some embodiments, the polysorbate is polysorbate-80.

In some embodiments, the pharmaceutical composition has a pH of about 5 to about 8. In some embodiments, the pharmaceutical composition has a pH of about 6 to about 7. In some embodiments, the pharmaceutical composition has a pH of about 5.5 to about 6.5. In some embodiments, the pharmaceutical composition has a pH of 5.5, 5.6, 5.7, 5.8, 5.9, 6.0, 6.1, 6.2, 6.3, 6.4, 6.5, 6.6, 6.7 or 6.8. In some embodiments, the pharmaceutical composition has a pH of 6.3.

In some embodiments, the pharmaceutical composition comprises about 10 mg/mL CM-101, about 10 mM L-Histidine, about 40 mM Glycine, about 4% (w/v) Sorbitol, about 0.01% (w/v) Polysorbate 80, and has a pH of about 6.3. In some embodiments, the pharmaceutical composition is for intravenous administration. In some embodiments, one vial of the pharmaceutical contains a fill volume of about 10.2 mL or about 10.3 mL, and/or a withdrawal volume of about 10 mL.

The present disclosure also provides a kit comprising a pharmaceutical composition comprising an anti-CCL24 monoclonal antibody. In some embodiments, the kit further comprises instructions for use. In some embodiments, the kit is configured to treat and/or prevent a fibrotic inflammatory disease in a subject. In some embodiments, the kit further comprises a device for administering the pharmaceutical composition. The present disclosure is not limited by the type of device utilized for administering the pharmaceutical composition. Indeed, a variety of devices are contemplated to be useful in a kit including, but not limited to, a skin applicator, a syringe, and/or an infusion device.

### Definitions

The terms “CCL24” or “eotaxin-2” are used interchangeably and refer to “Chemokine (C-C motif) ligand 24”, a cytokine belonging to the CC chemokine family which in human is encoded by the human CCL24 gene and located on human chromosome 7. CCL24 interacts with chemokine receptor CCR3. CCL24 activity includes induction of chemotaxis in eosinophils, basophils, T lymphocytes and neutrophils, as well as induction of angiogenic and migratory responses in endothelial and smooth muscle cells.

The term “pharmaceutically acceptable carrier, excipient or diluent” includes any and all solvents, dispersion media, coatings, antibacterial and antifungal agents and the like, as known in the art. The carrier can be solvent or dispersion medium containing, for example, water, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the therapeutic composition is contemplated.

By the term “prophylaxis” as herein defined it is meant to provide a “preventive treatment” or “prophylactic treatment”, namely acting in a protective manner, to defend against or prevent the appearance of a symptom of a disease, or disease onset or progression. In some embodiments, “prophylactic treatment” of a therapeutic agent is administered before the onset of the disease.

It is to be understood that the terms “treat”, “treating”, “treatment” or forms thereof, as used herein, mean reducing, preventing, curing, reversing, ameliorating, attenuating, alleviating, minimizing, suppressing or halting the deleterious effects of a disease or a condition or delaying the onset of one or more clinical indications of hepatic diseases, as defined herein.

The term “immunoconjugate” as herein defined refers to an antibody or any antigen-binding fragment thereof according to the disclosure that is conjugated (linked or joined) to an additional agent. Immunoconjugates may be prepared by any method known to a person skilled in the art, for example, by cross-linking the additional agent to the antibody according to the disclosure or by recombinant DNA methods.

The term “about” means a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

### EXAMPLES

### Example 1: Phase 1—Pharmacokinetics of IV Administration in Healthy Volunteers

PK analysis of the Phase 1 study was conducted to evaluate the pharmacokinetics of CM-101 following single intravenous infusion of 0.75, 2.5, 5 and 10 mg/kg CM-101 in healthy volunteers. The quantification of CM-101 in the plasma samples was performed using a validated ELISA-based assay. The lower limit of quantitation (LLOQ) value of CM-101 in the analyzed samples was 87.5 ng/mL. The pharmacokinetic analysis was performed using PK Solver 2.0 software that was validated against the SAS® software version 9.4 (SAS Institute, Cary, N.C., USA) and STATA SE Version 13.0 (STATA Corporation, Texas, USA).

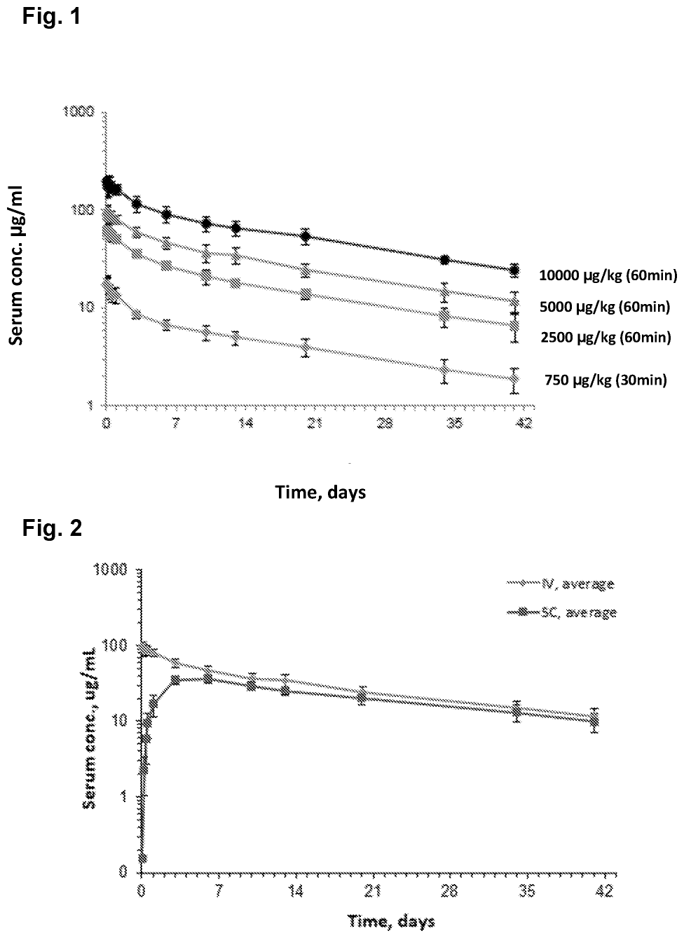

Following intravenous infusion to the healthy volunteers, CM-101 exhibited a biphasic serum concentration vs. time curve (rapid distribution phase, and slow elimination phase) (FIG. 1). The PK of CM-101 appears to be dose-proportional and the values of the PK parameters obtained in the non-compartmental and compartmental analysis of CM-101 concentration vs. time data are shown in Table 1.

CM-101 has a steady state volume of distribution of ˜80 mL/kg, and a terminal volume of distribution of ˜80-90 mL/kg. The clearance of CM-101 is ˜2.9-3.2 mL/kg/d. Target-mediated drug disposition (TMDD), or presence of anti-drug antibodies (neutralizing antibodies) was not evident in the analyzed concentration vs. time curves of CM-101, which exhibited linear terminal slope without apparent TMDD kinetics, or other concentration-dependent changes of the elimination kinetics. The terminal half-life of CM-101 was ˜19 days.

TABLE 1
Average pharmacokinetic parameters of CM-101 administered IV to healthy volunteers,
based on the non-compartmental pharmacokinetic analysis.

| Parameter | Unit | 0.75 mg/kg average | 0.75 mg/kg SD | 0.75 mg/kg % CV |
|---|---|---|---|---|
| Cₘₐₓ | μg/ml | 18.3 | 3.1 | 17.2 |
| Tₘₐₓ | d | 0.083 | 0.063 | 75.8 |
| AUC ₀₋ₜ | μg · d/ml | 190 | 29 | 15.4 |
| AUC _{0-∞} | μg · d/ml | 242 | 49 | 20.2 |
| AUC _{0-t/0-∞} | — | 0.791 | 0.042 | 5.3 |
| AUMC _{0-∞} | μg · d²/ml | 6274 | 2218 | 35.3 |
| MRT | d | 25.4 | 3.6 | 14.4 |
| K terminal | 1/d | 0.0374 | 0.0036 | 9.6 |
| t_{1/2} | d | 18.7 | 2.0 | 11.0 |
| V_{z} | mL/kg | 85.0 | 11.6 | 13.7 |
| Vₛₛ | mL/kg | 79.7 | 10.5 | 13.2 |
| CL | mL/kg/d | 3.19 | 0.61 | 19.2 |
| Cₘₐₓ/D | kg/mL | 0.0244 | 0.0042 | 17.2 |
| AUC _{0-∞}/D | kg · d/mL | 0.323 | 0.065 | 20.2 |
| Parameter | Unit | 2.5 mg/kg average | 2.5 mg/kg SD | 2.5 mg/kg % CV |
| Cₘₐₓ | μg/ml | 64.6 | 4.4 | 6.8 |
| Tₘₐₓ | d | 0.083 | 0.065 | 77.5 |
| AUC ₀₋ₜ | μg · d/ml | 708 | 77 | 10.9 |
| AUC _{0-∞} | μg · d/ml | 905 | 185 | 20.4 |
| AUC _{0-t/0-∞} | — | 0.795 | 0.075 | 9.4 |
| AUMC _{0-∞} | μg · d²/ml | 23848 | 10787 | 45.2 |
| MRT | d | 25.3 | 6.2 | 24.7 |
| K terminal | 1/d | 0.0377 | 0.0091 | 24 |
| t_{1/2} | d | 19.3 | 4.8 | 24.7 |
| V_{z} | mL/kg | 76.5 | 5.4 | 7.0 |
| Vₛₛ | mL/kg | 69.3 | 4.5 | 6.6 |
| CL | mL/kg/d | 2.85 | 0.55 | 19.4 |
| Cₘₐₓ/D | kg/mL | 0.0259 | 0.0018 | 6.8 |
| AUC _{0-∞}/D | kg · d/mL | 0.362 | 0.074 | 20.4 |
| Parameter | Unit | 5 mg/kg average | 5 mg/kg SD | 5 mg/kg % CV |
| Cₘₐₓ | μg/ml | 100.8 | 10.4 | 10.4 |
| Tₘₐₓ | d | 0.153 | 0.193 | 126.4 |
| AUC ₀₋ₜ | μg · d/ml | 1231 | 166 | 13.5 |
| AUC _{0-∞} | μg · d/ml | 1573 | 279 | 17.7 |
| AUC _{0-t/0-∞} | — | 0.789 | 0.059 | 7.5 |
| AUMC _{0-∞} | μg · d²/ml | 41614 | 15926 | 38.3 |
| MRT | d | 25.9 | 5.4 | 20.9 |
| K terminal | 1/d | 0.0365 | 0.0064 | 17.4 |
| t_{1/2} | d | 19.6 | 4.1 | 21.1 |
| V_{z} | mL/kg | 90.8 | 16.9 | 18.6 |
| Vₛₛ | mL/kg | 82.9 | 13.4 | 16.1 |
| CL | mL/kg/d | 3.27 | 0.66 | 20.2 |
| Cₘₐₓ/D | kg/mL | 0.0202 | 0.0021 | 10.4 |
| AUC _{0-∞}/D | kg · d/mL | 0.315 | 0.056 | 17.7 |
| Parameter | Unit | 10 mg/kg average | 10 mg/kg SD | 10 mg/kg % CV |
| Cₘₐₓ | μg/ml | 206.6 | 28.4 | 13.7 |
| Tₘₐₓ | d | 0.090 | 0.061 | 67.9 |
| AUC ₀₋ₜ | μg · d/ml | 2506 | 324 | 12.9 |
| AUC _{0-∞} | μg · d/ml | 3199 | 368 | 11.5 |
| AUC _{0-t/0-∞} | — | 0.784 | 0.059 | 7.5 |
| AUMC _{0-∞} | μg · d²/ml | 84870 | 21703 | 25.6 |
| MRT | d | 26.4 | 4.9 | 18.7 |
| K terminal | 1/d | 0.0369 | 0.0076 | 20.6 |
| t_{1/2} | d | 19.5 | 4.3 | 22.2 |
| V_{z} | mL/kg | 88.4 | 18.4 | 20.8 |
| Vₛₛ | mL/kg | 83.1 | 15.5 | 18.7 |
| CL | mL/kg/d | 3.16 | 0.36 | 11.5 |
| Cₘₐₓ/D | kg/mL | 0.0207 | 0.0028 | 13.7 |
| AUC _{0-∞}/D | kg · d/mL | 0.320 | 0.037 | 11.5 |

| Parameter | P value |
|---|---|
| Cₘₐₓ | NC |
| Tₘₐₓ | 0.920 |
| AUC ₀₋ₜ | NC |
| AUC _{0-∞} | NC |
| AUC _{0-t/0-∞} | NC |
| AUMC _{0-∞} | NC |
| MRT | 0.956 |
| K terminal | 0.998 |
| t_{1/2} | 0.998 |
| V_{z} | 0.270 |
| Vₛₛ | 0.085 |
| CL | 0.723 |
| Cₘₐₓ/D | 0.015^{a} |
| AUC _{0-∞}/D | 0.723 |

P value-of the non-parametric Kruskal-Wallis ANOVA test
NC-not calculated (for the parameters with dose-dependent values, in drugs with linear pharmacokinetics)
^{a}statistically-significant differences between 2.5 mg/kg vs. 5 mg/kg groups were identified using Dunn's post-test.

### Example 2: Phase 1—Pharmacokinetics of SC Administration in Healthy Volunteers

PK analysis of the Phase 1 study was conducted to evaluate the pharmacokinetics of CM-101 following single subcutaneous injection of 5 mg/kg CM-101 in healthy volunteers. The quantification of CM-101 in the plasma samples was performed using the same methodology used for the phase 1 IV administration in healthy volunteers.

The values of the pharmacokinetic parameters obtained in the non-compartmental analysis of CM-101 concentration vs. time data are shown in Table 2 and CM-101 has a steady state apparent volume of distribution of ˜134 mL/kg, and the terminal volume of distribution of ˜131 mL/kg. The terminal half-life of CM-101 was ˜20.8 days and the apparent clearance of ˜4.5 mL/kg/d.

Comparison of the pharmacokinetic data of 5 mg/kg of CM-101 using IV administration to the SC administration indicates consistent pharmacokinetic behavior of CM-101 in these studies (FIG. 2). Based on the AUC values in the IV and SC studies, the absolute bioavailability of CM-101 following subcutaneous injection was approximately 75%.

TABLE 2
Average pharmacokinetic parameters of 5 mg/kg CM-101 administered to healthy
volunteers, based on the non-compartmental pharmacokinetic analysis.

| Parameter | Unit | V101 | V103 | V104 | V105 | V106 | V107 | average | SD | % CV |
|---|---|---|---|---|---|---|---|---|---|---|
| Cₘₐₓ | μg/mL | 31.2 | 30.2 | 37.7 | 37.0 | 39.8 | 42.3 | 36.4 | 4.8 | 13 |
| Tₘₐₓ | d | 3.0 | 6.0 | 3.0 | 3.0 | 6.0 | 6.0 | 4.5 | 1.6 | 37 |
| AUC₀₋ₜ | μg · d/mL | 711 | 683 | 926 | 820 | 1029 | 938 | 851 | 137 | 16 |
| AUC_{0-∞} | μg · d/mL | 854 | 915 | 1361 | 1101 | 1547 | 1180 | 1160 | 264 | 23 |
| AUC_{0-t/0-∞} | — | 0.83 | 0.75 | 0.68 | 0.74 | 0.66 | 0.79 | 0.74 | 0.06 | 9 |
| AUMC_{0-∞} | μg · d²/mL | 20181 | 27479 | 48839 | 33100 | 58792 | 30904 | 36549 | 14421 | 39 |
| MRT | d | 23.6 | 30.0 | 35.9 | 30.1 | 38.0 | 26.2 | 30.6 | 5.5 | 18 |
| k terminal | 1/d | 0.0451 | 0.0339 | 0.0283 | 0.0328 | 0.0267 | 0.0401 | 0.0345 | 0.0070 | 20 |
| t_{1/2} | d | 15.4 | 20.5 | 24.5 | 21.1 | 25.9 | 17.3 | 20.8 | 4.0 | 19 |
| Vₛₛ/F | mL/kg | 138.3 | 164.3 | 131.9 | 136.4 | 122.8 | 111.0 | 134.1 | 17.9 | 13 |
| V_{z}/F | mL/kg | 129.7 | 161.4 | 129.7 | 138.4 | 120.8 | 105.7 | 131.0 | 18.6 | 14 |
| CL/F | mL/kg/d | 5.85 | 5.47 | 3.67 | 4.54 | 3.23 | 4.24 | 4.50 | 1.01 | 22 |

### Example 3: Phase 1b—Pharmacokinetics of Repeated 2.5 mg/kg IV Administration, in NAFLD Patients

PK analysis of the data from the completed 2.5 mg/kg dose cohort of an ongoing Phase 1b study was conducted to evaluate the pharmacokinetics of CM-101 following multiple IV infusion of 2.5 mg/kg CM-101 in NAFLD patients. The quantification of CM-101 in the plasma samples was performed using the same methodology used for the phase 1 IV and SC administration studies in healthy volunteers.

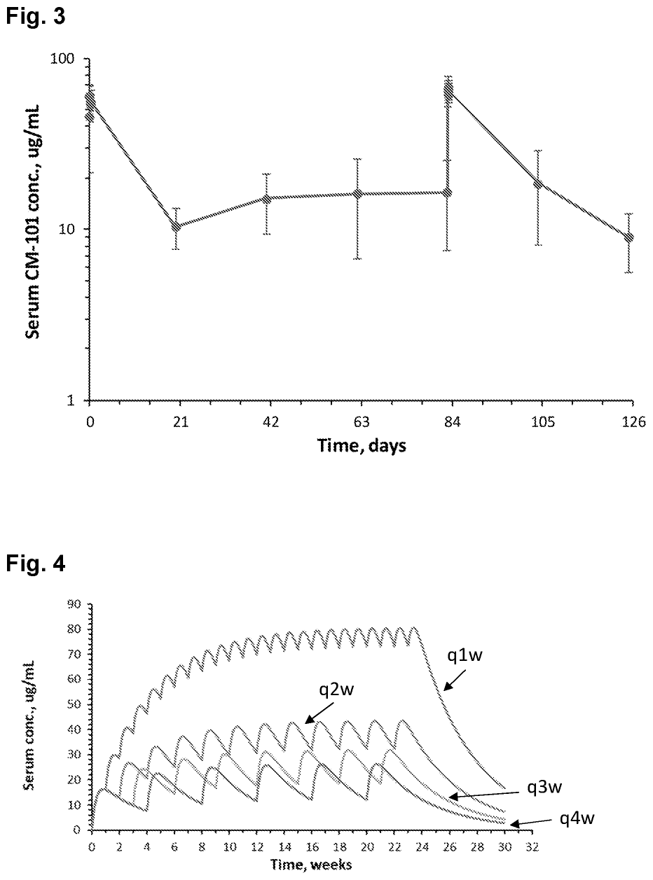

The shape of the CM-101 serum concentration vs. time curves in the individual patients was similar, with stable trough (days 20, 41, 62, 83, 104) and peak (days 0 and 83) concentrations of CM-101 (repeated intravenous infusions of 2.5 mg/kg CM-101 over ˜1 hr, at 3-weeks intervals) (FIG. 3).

In this population of NAFLD patients, CM-101 apparently exhibited a bi-phasic pharmacokinetic behavior that was appropriately described using a classical two-compartmental pharmacokinetic model. Kinetically, the obtained shape of the serum concentration vs. time curves apparently indicates gradual distribution kinetics of CM-101 in the body (over ˜4.5 days; 4 half-lives of Alpha; Table 3) and much slower elimination kinetics (over ˜81 days; 4 half-lives of Beta; Table 3).

CM-101 antibody has the steady state volume of distribution (volume of the central compartment,) of ˜45.8 mL/kg, the volume of the peripheral compartment of ˜108 mL/kg, and the terminal volume of distribution of ˜119 mL/kg (Table 3).

The clearance of CM-101 is ˜4.27 mL/kg/d (Table 3). Target-mediated drug disposition (TMDD), or presence of anti-drug antibodies (neutralizing antibodies) was not evident in the analyzed concentration vs. time curves of CM-101, which showed no obvious concentration-dependent changes of the elimination kinetics, or changes in the trough levels upon repeated administrations.

TABLE 3
Pharmacokinetic parameters of CM-101 following five administrations
of 2.5 mg/kg administered IV Q3W

| | | 2.5 mg/kg IV five | | |
|---|---|---|---|---|
| | | administrations Q3W | | |
| Parameter | Units | Average | SD | % CV |
| A | μg/mL | 0.0143 | 0.0011 | 7 |
| B | μg/mL | 0.00763 | 0.00094 | 12 |
| Alpha | 1/d | 0.615 | 0.013 | 2 |
| Beta | l/d | 0.0355 | 0.0068 | 19 |
| t_{1/2} Alpha | d | 1.13 | 0.02 | 2 |
| t_{1/2} Beta | d | 20.3 | 4.8 | 23 |
| V₁ | mL/kg | 45.8 | 3.9 | 8 |
| V₂ | mL/kg | 62.4 | 6.2 | 10 |
| Vₛₛ | mL/kg | 108.2 | 10.0 | 9 |
| V_{z} | mL/kg | 119.4 | 12.2 | 10 |
| CL | mL/kg/d | 4.27 | 1.05 | 25 |

* Calculated from the MONOLIX output using the Convert.xls tool

### Example 4: Simulation of Pharmacokinetics of CM-101 with IV Administration

Using the exposure data from human studies (single administration phase I study with IV and SC administration up to 10 mg/kg, as well as the repeated dose administration with 2.5 mg/kg IV administration), different simulations using combinations of the available data were performed to assess the expected exposure levels of IV administration of CM-101 given once every 3 weeks at a dose of 10 mg/kg (Table 4). In all three simulations (using different compositions of the available data) the expected average CM-101 concentrations range from 117-150 ug/mL and the maximal predicted values range from 290-295 ug/mL.

TABLE 4
Pharmacokinetic simulations of CM-101 exposure following up
to eight administrations of 10 mg/kg administered IV Q3W

| | Data used in the PK Simulation | | |
|---|---|---|---|
| | | | IV & SC Single |
| | | | Admin' studies |
| | IV & SC Single | Multiple Admin' | & Multiple |
| | Admin' Studies | IV study | Admin' IV study |
| | Simulated | Simulated | Simulated |
| | CM-101 | CM-101 | CM-101 |
| | concentrations | concentrations | concentrations |
| | ug/mL | ug/mL | ug/mL |
| | Average | Average | Average |
| | (Min; Max) | (Min; Max) | (Min; Max) |
| I.V, 10 mg/kg Q3W | 150 | 117 | 147 |
| for 8 administrations | (90; 295) | (71; 290) | (87; 293) |

### Example 5: Simulation of Pharmacokinetics of CM-101 with SC Administration

Using the exposure data from human studies (single administration phase I study with IV and SC administration up to 10 mg/kg, as well as the repeated dose administration with 2.5 mg/kg IV administration), different simulations using combinations of the available data were performed to assess the expected exposure levels of SC administration of CM-101 given once every 1-4 weeks at a dose of 2.5 or 5 mg/kg (Table 5). In both simulations (using different compositions of the available data) the expected average CM-101 concentrations were 78-79 ug/mL and the maximal predicted values of 87-88 ug/mL.

Simulation of prolonged repeated dosing of CM-101 at 2.5 mg/kg CM-101 Q3W indicated average predicted peak concentrations of CM-101 of 73.5 ug/mL, which correlates to the average predicted exposure in the 5 mg/kg repeated dosing—and was already seen to be safe in patients.

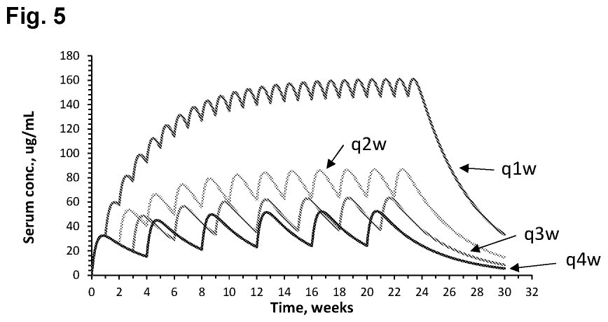

FIGS. 4 and 5 depicts a simulation of CM-101 serum concentration over time for the different simulated doses of SC administration. The predicted exposure of CM-101 following up to 24 repeated (Q1W, Q2W, Q3W, Q4W) administrations of 2.5 or 5 mg/kg CM-101 SC reached up to 80 and 160 ug/ml exposure at steady state, respectively

TABLE 5
Pharmacokinetic simulations of CM-101 exposure following up to
ten administrations of 5 mg/kg administered SC Q2W

| | Data used in the PK Simulation | |
|---|---|---|
| | | IV & SC Single Admin' |
| | IV & SC Single | studies & Multiple |
| | Admin' Studies | Admin' IV study |
| | Simulated | Simulated |
| | CM-101 concentrations | CM-101 concentrations |
| | ug/mL | ug/mL |
| | Average | Average |
| | (Min; Max) | (Min; Max) |
| S.C, 5 mg/kg | 79 | 78 |
| Q2W for 10 | (64, 88) | (64, 87) |
| administrations | | |

### Example 6: Treatment for PSC

The study is a Phase 2a, randomized, double-blind, placebo-controlled, study aimed to evaluate the safety and efficacy of CM-101 in adult subjects with large duct PSC as their predominant disease. Recruited subjects will be ones with serum alkaline phosphatase (ALP) of at least 1.5 times the upper limit of normal (×1.5 ULN). Subjects with concomitant IBD are eligible for recruitment as long as their disease is stable (defined as Partial Mayo score of ≤3 for Ulcerative Colitis (UC) patients, or CDAI<150 for Crohn's Disease (CD) patients) and the absence of high-grade dysplasia in colonic biopsies within 18 months of Randomization, or a history of colectomy (at any time). Up to 45 subjects will be randomized to receive 10 mg/kg CM-101, or placebo, in a 2:1 ratio. Randomization will be stratified based on ongoing Ursodeoxycholic Acid (UDCA) treatment. Subjects will receive a dose of investigational product once every 3 weeks for a total of 5 administrations resulting in a total coverage of 15 weeks. The suggested study design, duration of treatment and patient population is adequate for a phase 2a study in PSC that evaluates changes in ALP and ELF score as its primary endpoints.

The selected dose for the proposed study is 10 mg/kg, by IV administration once every 3 weeks, Administration of CM-101 according to the selected dose and administration schedule will demonstrate clinical benefit and safety in PSC patients.

### Example 7: Treatment for NASH

The study is a Phase 2a, randomized, double-blind, placebo-controlled, study aimed to evaluate the safety and efficacy of CM-101 in adult subjects with NASH as their predominant disease. Up to 40 subjects will be randomized to receive 5 mg/kg CM-101 (n=25), or placebo (n=15). Recruited subjects will receive 8 CM-101 or matching placebo administrations Q2W providing treatment coverage of 17 weeks. The suggested study design, duration of treatment and patient population is adequate for a phase 2a study in NASH that evaluates safety as its primary endpoints.

The selected dose for the proposed study is 5 mg/kg, by SC administration once every 2 weeks, Administration of CM-101 according to the selected dose and administration schedule will demonstrate clinical benefit and safety in NASH patients.

### Example 8: Phase I Single IV Administration Study in Healthy Volunteers

This single-center, randomized double-blind, placebo-controlled, single-dose, dose-escalation study in healthy volunteers was designed to evaluate the safety, tolerability and pharmacokinetics of a single IV dose of CM-101 in 32 healthy male volunteers. The study included four (4) escalating dose groups, with eight (8) subjects each, the follow-up period for each subject is 42 days. The tested doses are 0.75, 2.5, 5 and 10 mg/kg. In each Dose Group subjects were randomized in 3:1 ratio to receive a single IV infusion of CM-101 (n=6) or matching placebo (n=2).

In the phase I single administration study, healthy volunteers were screened for up to 28 days prior to drug administration. The study included 4 escalating dose groups of 8 subjects each. The initial CM-101 dose was 0.75 mg/kg followed by single doses of 2.5, 5 and 10 mg/kg administered to subsequent groups.

In each dose group subjects were randomized in a 3:1 ratio to receive a single IV infusion of either CM-101 (n=6) or placebo (n=2). For safety considerations, in all dose groups the first two subjects (one receiving active drug and one placebo in a blinded manner) were dosed initially, followed by the rest of the group (dosing of the rest of the dose group was done at least 14 days after the first two subjects had been dosed).

Each subject received the IV infusion of the investigational drug or placebo in the morning of the dosing day (Day 1). The subjects remained under medical supervision at the Phase I Unit for approximately 24 hours post-dose and underwent safety, PK and PD evaluations. Seven additional ambulatory follow-up visits took place over the 42 days follow-up period.

A total of 32 subjects were enrolled into the study and randomized to 4 treatment groups (0.75 mg/kg, 2.5 mg/kg, 5.0 mg/kg, 10 mg/kg) and 8 subjects in the placebo group. All 32 subjects completed the study as planned.

Blood samples to evaluate hs-CRP levels were collected pre-dose, and on days 7 and 35. The hs-CRP levels were within normal range and no changes over time were detected.

### Example 9: Phase I Single SC Administration Study in Healthy Volunteers

This single-center, randomized double-blind, placebo-controlled, single-dose study in healthy volunteers was using a design similar to the IV SAD study (protocol CM-101-I-001). The study evaluated the safety, tolerability and pharmacokinetics of a single SC dose of CM-101 healthy male volunteers. In the CM-101 5 mg/kg dose subjects were randomized in 3:1 ratio to receive a single SC injection of CM-101 (n=6) or matching placebo (n=2).

In the phase I single administration study, healthy volunteers were screened for up to 28 days prior to drug administration. The study included evaluation of one dose group. Subjects were randomized in a 3:1 ratio to receive a single SC injection of either CM-101 5 mg/kg (n=6) or matching placebo (n=2).

Each subject received the SC injection of the investigational drug or placebo in the morning of the dosing day (Day 1). The subjects remained under medical supervision at the Phase I Unit for approximately 24 hours post-dose and underwent safety, PK and PD evaluations. Seven additional ambulatory follow-up visits took place over the 42 days follow-up period.

A total of 8 subjects were enrolled into the study and randomized to receive CM-101 5.0 mg/kg as SC injection (n=6) or placebo (n=2). All 8 subjects completed the study as planned.

### Example 10: Phase Ib MAD Study, IV/SC Administration in NAFLD Patients

This study includes evaluation of two dose levels. First dose level of 2.5 mg/kg CM-101 administered as IV infusions followed by the second dose level of 5 mg/kg CM-101 dose administered as slow SC injections. Both dose levels involve 5 drug administrations over 12 weeks (Q3W) providing 15 weeks of treatment coverage. In both dose levels subjects are randomized in a 3:1 ratio to receive either CM-101 (n=6) (2.5 mg/kg IV or 5 mg/kg SC) or matching placebo (n=2).

From this study we report interim safety data of the already completed 2.5 mg/kg dose group treated using IV infusion.

### Subject Baseline Characteristics and Demographics

The 8 recruited subjects included two (2) females and six (6) males. The main medical history findings were disease related to the NAFLD population

### Example 11: Formulation of CM-101

An initial high-dose formulation feasibility study resulted in CM101 concentrations greater than 150 mg/mL in histidine and phosphate buffered (pH 6.1-6.9) solutions containing varying amounts of arginine and glutamic acid along with either sorbitol or sodium chloride. Single-phase, clear, free-flowing, light yellow solutions were observed in all formulations. Osmolality of solutions ranged from 306-365 mOsm. Importantly, viscosity levels were quite low for high-dose formulations and ranged from 6.4-10.7 mPa*s, in which lower values were detected in the formulations containing sodium chloride as opposed to sorbitol as the tonicity adjuster. Phosphate buffered formulations tended to have slightly lower purities than the corresponding histidine buffered formulations by SE-HPLC. Slightly lower turbidity values were observed at low pH (pH 6.1) as opposed to pH 6.5 and above. A final formulation selection study evaluated four CM101 formulations at a concentration of 175 mg/mL in histidine buffered formulations. Following twelve weeks of storage at −70° C., 2-8° C., and 25° C., SE-HPLC purity levels are consistent with t=0 values for all formulations. Calculation of the rate of impurity formation, as determined from the SE-HPLC data, suggested solution stability was calculated to be approximately 3800 days—or approximately >10 years—in the CM101 pH 6.1 formulation which contained 150 mM arginine and 150 mM glutamic acid. In the CEX-HPLC analyses, a clear stability preference was found for the pH 5.8 and 6.0 ADU-1604 formulations as compared to the higher pH formulations. Calculation of the rate of impurity formation, as determined from the CEX-HPLC data, suggested solution calculated to be on the order of 600 days—or approximately 1.7 years—in the CM101 pH 6.1 formulation containing 150 mM arginine and 150 mM glutamic acid prior to reaching 95% of the original sample purity. It should be noted that no formal limit for the variants has been established and this will need to be correlated to potency data as well to set an acceptable limit of changes in the charged species. Relative potency values were within assay variability (20%) for each of the formulations exposed to temperature (−70° C., 2-8° C., 25° C.) as well as freeze-thaw and shaking conditions. No clear trends or changes in potency were observed. Overall, the 10 mM histidine, 150 mM arginine, 150 mM glutamic acid, 0.02% PS80, pH 6.1, CM101 formulation provides optimal stability. Table 6 shows the formulations tested in the study.

TABLE 6
CM-101 formulation tests

| | Formu- | | Argi- | | Sor- | | |
|---|---|---|---|---|---|---|---|
| | lation | | nine | Glutamic | bitol | NaCl | 0.01% |
| | ID | pH | (mM) | Acid (mM) | (%) | (mM) | PS80 |
| 10 mM | #1 | 6.1 | 150 | 150 | | | + |
| Histidine | #2 | 6.5 | 55 | 55 | 3 | | + |
| | #3 | 6.7 | 55 | 55 | 3 | | + |
| | #4 | 6.5 | 55 | 55 | | 80 | + |
| | #5 | 6.9 | 55 | 55 | | 80 | + |
| 10 mM | #6 | 6.1 | 150 | 150 | | | + |
| Potassium | #7 | 6.5 | 55 | 55 | 3 | | + |
| phosphate | #8 | 6.7 | 30 | 30 | 4 | | + |
| | #9 | 6.5 | 55 | 55 | | 90 | + |
| | #10 | 6.9 | 55 | 55 | | 90 | + |

| | | | |
|---|---|---|---|
| Control | #11 | 6.3 | 10 mM histidine, 4% sorbitol, 40 mM |
| formulation | | | glycine, 0.01% PS80, pH 6.3 |

### Example 12: Pharmacodynamic Assessment of Total CCL24 Sera Levels Following Treatment with CM-101 in Phase 1B (Cohort 1) Clinical Trial

Study Design: The purpose of this study is to assess the pharmacodynamic properties of CM-101 by evaluating the total sera levels of CCL24 in NAFLD patients following five IV administration of CM-101, as compared to placebo treated group. Eight stable NAFLD subjects that are in general good health were screened for up to 28 days prior to drug administration. The study included five infusion of CM-101 2.5 mk/kg once every three weeks, equal to 15 weeks of drug coverage and followed by 21 days of safety follow-up. Subjects were randomized in a 3:1 ratio to receive five IV infusion of either CM-101 (n=6) or placebo (n=2).

Sample Processing: A blood sample of 15 mL was taken on dosing day 1 (TRT1) and all remaining dosing days (TRT 2-5) at pre-dose time point. An additional 5 mL blood sample was taken at 2 hours post dose in order to determine pharmacodynamic parameters. 15 mL blood samples was also taken on follow-up visits 1 and 2 (Visit 7/End of treatment (EOT) and Visit 8/End of study (EOS). Immediately after sample is drawn, the tube was gently inverted 180° and back, 5-6 times. Samples were allowed to clot in an upright position for a minimum of 30 minutes but no longer than 2 hours. Samples were centrifuge for 15 minutes at 1000×g at room temperature (18-25° C.). Serum samples, from each time point, were aliquoted using a disposable pipette in 2×2 ml cryovials with at least 0.5 mL in the first cryovial and the rest in the second cryovial. The samples were labeled with a designated label, which included the following details:

Measuring Total levels of human CCL24: CCL24 levels in serum samples were tested following feasibility and validation. Total CCL24 was measured in the following time points: pre-dose and 2 hours post dose for treatment visits 2-5, as well as EOT and EOS visits as well.

Results:

NAFLD patients were administered with 2.5 mg/kg CM-101 or matching placebo using IV infusion every 3 weeks for 5 consecutive treatments. To evaluate target engagement of the CM-101 antibody with its target, CCL24, we measured CCL24 levels in the circulation. Total CCL24 levels that represents the levels of both free CCL24 and CM-101-bound CCL24 were assessed at pre- and post-drug administration time points.

Engagement with an antibody results in sequestration of the chemokine from the intravascular space to form the antibody-target complexes and induce increased total levels of the chemokine in the circulation. The generation of these complexes blocks chemokine activity thereby resulting in reduced signaling and inhibition of its effects. These complexes follow the kinetic pattern of the antibody and overtime are eliminated/cleared from the circulation, reducing target availability. Upon multiple administrations, a new equilibrium of target production and complex clearness is formed, dictating reduced target availability for biological activity.

CM-101 treatment resulted in increased CCL24 levels in the circulation, evaluated by a validated ELISA assay that recognizes both free CCL24 and CM-101-CCL24 complexes (i.e. total CCL24 level). In each administration of CM-101, the magnitude of elevation of newly formed complexes in the circulation is proportional to available free CCL24.

### Example 13: Pharmacodynamic Assessment in Phase 1 Clinical Trial of Total CCL24 Sera Levels Following Treatment with CM101

Study design: The purpose of this study is to assess the pharmacodynamic properties of CM-101 by evaluating the total sera levels of CCL24 in healthy volunteers following a single IV administration of CM-101, as compared to placebo treated group. 32 Healthy volunteers were screened for up to 28 days prior to drug administration. The study included 4 escalating dose groups of 8 subjects each. The initial CM-101 dose was 0.75 mg/kg followed by single doses of 2.5, 5 and 10 mg/kg administered IV to subsequent groups. In each of the dose groups subjects were randomized in a 3:1 ratio to receive a single IV infusion of either CM-101 (n=6) or placebo (n=2). Altogether 24 subjects received the drug (6 per cohort) and 8 subjects received placebo.

Sample processing: Blood samples (20 mL) were collected at the following time points: on dosing day: at pre-dose, 1 hour, 8 hours and 24 hours after the end of infusion (all post-infusion blood were drawn within AO min of the assigned time point), and on Day 4, Day 7, Day 11, Day 14 and Day 21, day 35 and day 42. Immediately after sample is drawn, the tube was gently inverted 180° and back, 5-6 times. Samples were allowed to clot in an upright position for a minimum of 30 minutes but no longer than 2 hours. Samples were centrifuge for 15 minutes at 1000×g at room temperature (18-25° C.).

Experiment time points are: Pre-dose; 8 hours; Day 4; Day 11; Day 21; Day 35; Day 42

Measurement of total levels of human CCL24: CCL24 levels in serum samples were tested b following feasibility and validation. Total CCL24 was measured in following time points: pre-dose, 8 hours, 4 days, 11 days, 21 days, 35 days and 42 days. The average mean of the percentage change was calculated separately for each dosing group: 0.75, 2.5, 5 and 10 mg/kg and for the placebo treated volunteers.

Results:

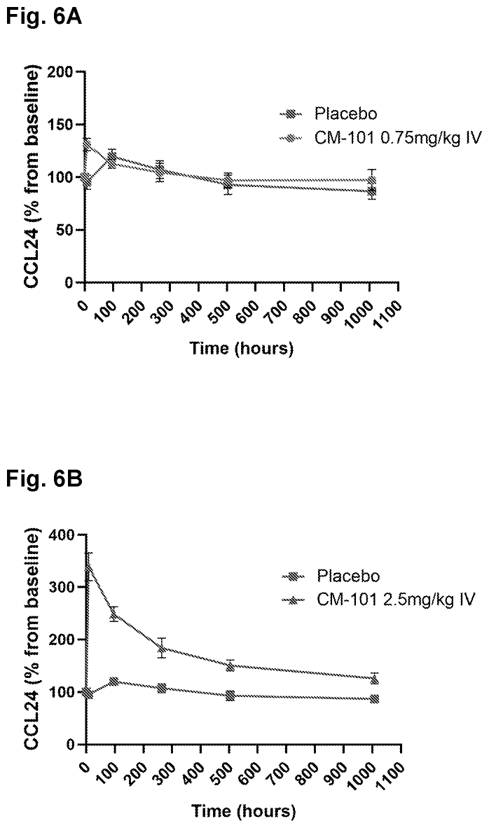

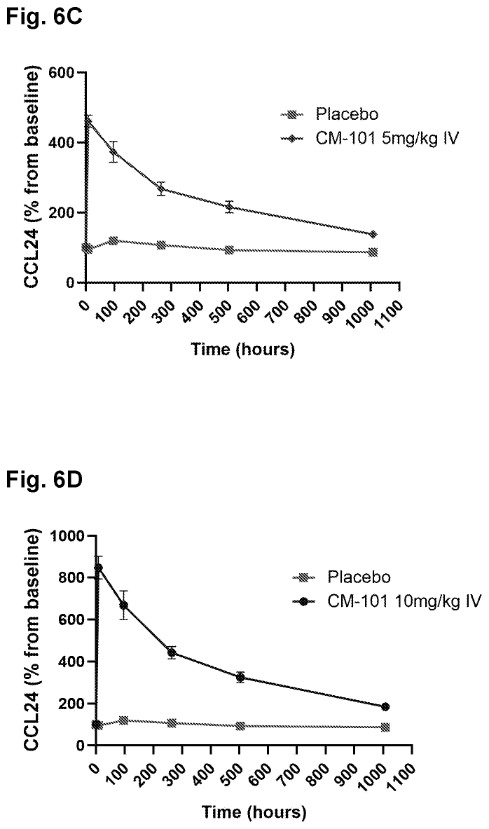

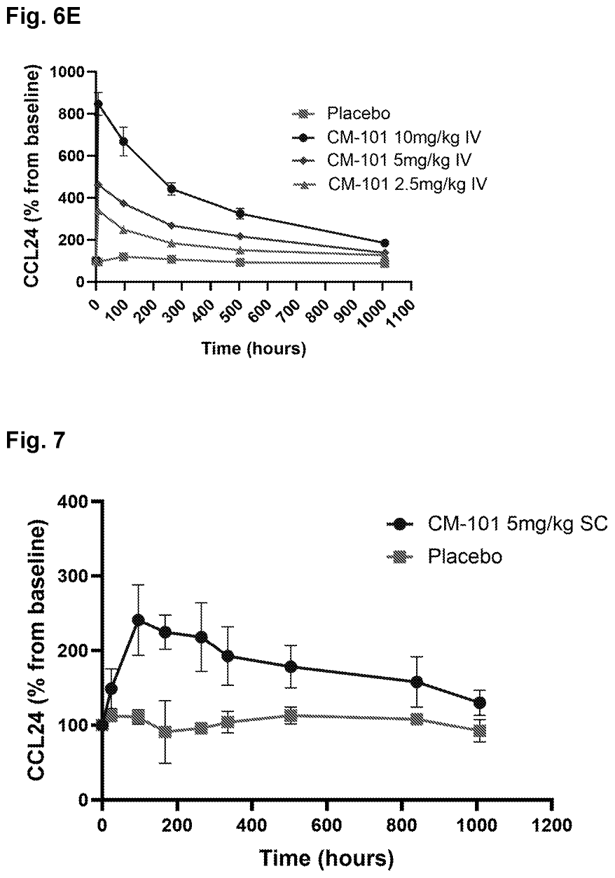

During the study CM-101 was administered at four escalating doses: 0.75, 2.5, 5 and 10 mg/kg. Each cohort included 6 subjects receiving the drug and 2 subjects receiving placebo. Overall 24 subjects received a single administration of the drug (6 per cohort) and 8 subjects received placebo. The total CCL24 levels represents the levels of both free CCL24 and CM-101-bound CCL24. Assessing the levels of total CCL24 levels in different time points pre and post drug administration revealed a significant dose dependent increase of total CCL24 levels, due to the binding of CM-101 to CCL24 in the circulation and its sequestration from the intravascular space. Total CCL24 levels reached a peak after 8 hours then decreased gradually until the last time point tested (day 42). CCL24 total levels reached an average peak (change from baseline) elevation after 8 hours that was dose dependent in subjects receiving 0.75, 2.5, 5 and 10 mg/kg, respectively (FIGS. 6A-6E).

### Example 14: Pharmacodynamics Assessment of Total CCL24 Sera Levels Following Subcutaneous Treatment with CM-101

Study design: The purpose of this study is to assess the pharmacodynamics properties of CM-101 by evaluating the sera levels of CCL24 in healthy volunteers following a single SC administration of CM-101, as compared to placebo treated group. 8 Healthy volunteers were screened for up to 28 days prior to drug administration. The study included one dose group of 8 subjects. The CM-101 dose was 5 mg/kg administered SC. Subjects were randomized in a 3:1 ratio to receive a single SC of either CM-101 (n=6) or placebo (n=2).

Sample processing: Sampling of whole blood was performed in three 5 mL (total 15 ml) Serum-Separating Tubes (SST tubes) (no additives) in the following time points: pre dose, 24 hours, 4 days, 7 days, 11 days, 14 days, 21 days, 35 days, 42 days. Immediately after sample is drawn, the tube was gently inverted 180° and back, 5-6 times. Samples were allowed to clot in an upright position for a minimum of 30 minutes but no longer than 2 hours. Samples were centrifuged for 15 minutes at 1000×g at room temperature (18-25° C.). Serum samples, from each time point, were aliquoted using a disposable pipette in 2×2 ml cryovials with at least 0.5 mL in the first cryovial and the rest in the second cryovial.

Experiment time points: Pre-dose; 24 hours; Day 4; Day 7; Day 11; Day 14; Day 21; Day 35; Day 42.

Measurement of total levels of human CCL24: CCL24 levels in serum samples were tested following reestablishment of validation parameters and QCs in the following time points: pre-dose, 24 hours, 4 days, 7 days, 11 days, 14 days, 21 days, 35 days and 42 days.

CCL24 total levels were calculated as change from baseline (pre-dose) for each individual.

Results:

During the study a single injection of 5 mg/kg CM-101 was administered to healthy volunteers. The cohort included 6 subjects receiving the drug and 2 subjects receiving placebo. The total CCL24 levels represents the levels of both free CCL24 and CM-101-bound CCL24. Assessing the levels of total CCL24 levels in different time-points pre and post drug administration revealed a significant increase of CCL24 levels reflecting the binding of CM-101 to CCL24 in the circulation. Total CCL24 levels (FIG. 7) reached a maximum exposure four days post treatment. Gradually reduction of total CCL24 levels is seen from day 7 until the last time point tested at day 42. In placebo treated healthy volunteers CCL24 total levels in sera remained steady during the study.

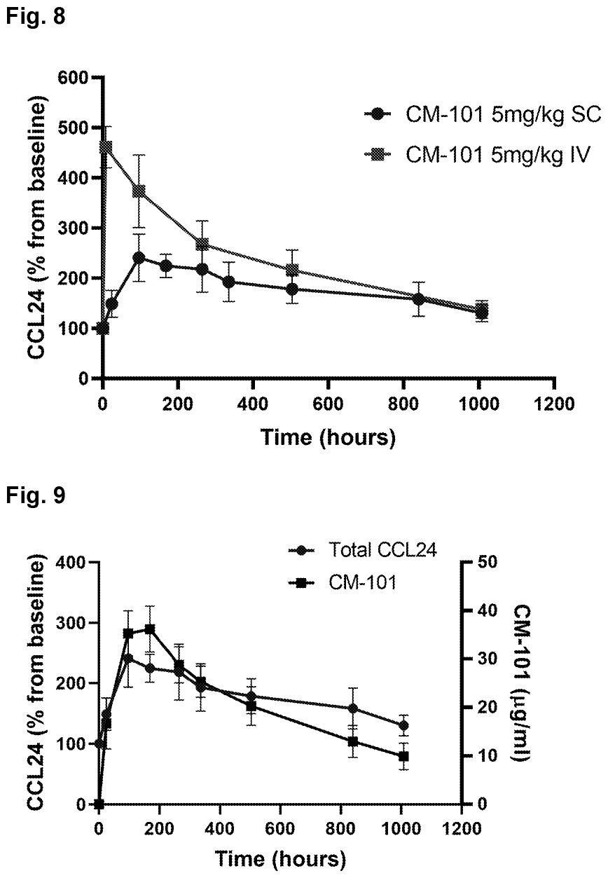

CM-101 is available in two distinct formulations allowing either intravenous (IV) or subcutaneous (SC) administration. Based on the data gathered in a previous clinical phase 1 study using the IV formulated drug (CM-1014-001), we were able to compare CCL24 total levels in the serum of healthy volunteers dosed with either 5 mg/kg CM-101 administered by infusion (IV formulation) or by injection (SC formulation) (FIGS. 8 and 9). When using the IV administration, total CCL24 increased very rapidly, within a few hours post dosing, followed by gradual decrease of CCL24 along time (dependent of the PK profile). Using the SC administration, the elevation of total CCL24 was more gradual achieving a peak 4-7 days post dosing that remained stable until day 11, followed by a gradual decreased (PK dependent). When comparing the area under curve (AUC) for both administrations we find a very similar pattern between the target engagement and bioavailability using the SC administration compared to the IV, mostly attributed to the rapid increase using IV compared to moderate increase using SC (Table 7). The table is showing the calculation of average AUC of 6 healthy volunteers treated with 5 mg/kg CM-101 either by IV or SC administration.

TABLE 7
Comparison of AUC of total CCL24 levels in healthy volunteers
treated with a single dose of 5 mg/kg CM-101 IV or SC administration.

| | CM-101 5 mg/kg SC | CM-101 5 mg/kg IV |
|---|---|---|
| Baseline | 0 | 0 |
| Total Area | 181760 | 239894 |
| Std. Error | 10022 | 15588 |
| 95% Confidence Interval | 162118 to 201402 | 209341 to 270447 |

### Example 15: CM-101 Demonstrates Reduction in Serum Fibrotic Biomarkers in a Phase 1b Randomized, Controlled Multiple Dose Trial in NAFLD Patients

Methods:

This controlled randomized double-blind single-center Phase 1b trial assessed safety, tolerability, PK, anti-drug antibodies and exploratory pharmacodynamics of multiple CM-101 administrations in 16 NAFLD patients with normal liver enzymes (see Example 10 above). The mean age of the treated subjects was 49.5±10.8 years (56% males) with similar demographics and laboratory parameters in study groups. Patients received five CM-101 treatments (every 3 weeks), either IV 2.5 mg/kg (6 vs. 2 matching placebo cases) or SC 5 mg/kg (6 vs. 2) and had a post treatment follow-up of 42 days analyzing non-invasive fibrotic parameters and PK. Namely, serum samples were analyzed for collagen turnover markers (Pro-C3, Pro-C4, C3M) and fibrotic biomarkers (TIMP-1, TIMP-2, PDGF AA) using multiplex (Millipore). Pharmacokinetic (PK) was measured by ELISA for CM-101 serum levels. Elastography was preformed using Fibroscan.

Results:

Five repeated administrations of CM-101 were found to be safe and well tolerated for both doses and administration modes. All reported adverse events were mild or moderate (no severe adverse events (SAE) were reported). One non-drug related SAE was reported in the SC dose group (meningioma) that led to early patient discontinuation. No injection site reactions were reported for both SC and IV administrations.

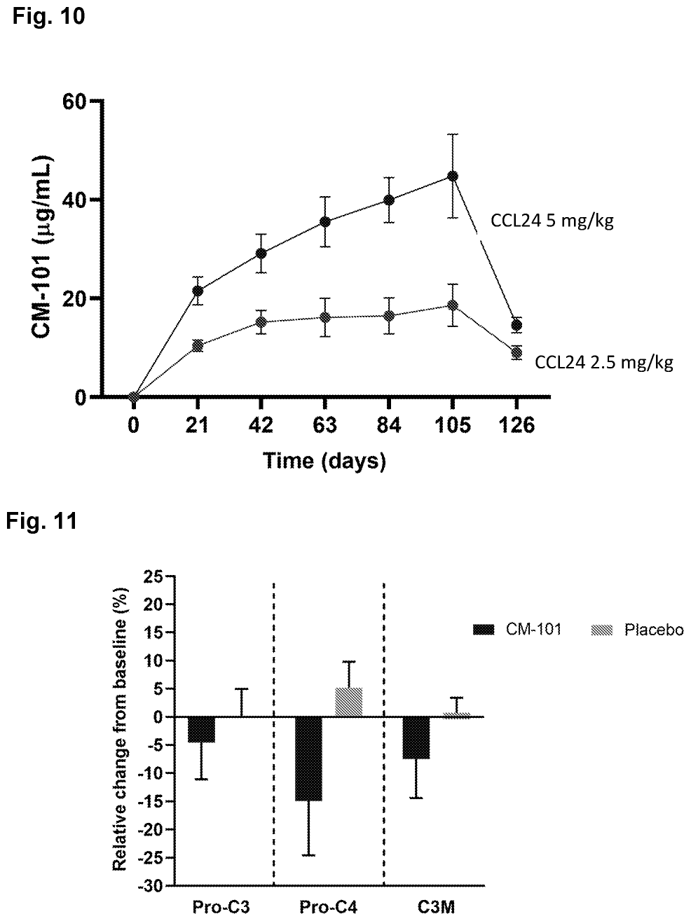

Following treatment, a dose proportional increase in serum CM-101 levels was shown (FIG. 10).

Moreover, following CM-101 treatment (with either dose), reduction in collagen turnover and fibrotic biomarkers was noted. The data for both doses was pooled and for each marker change from baseline (%) is presented as median value±IQR.

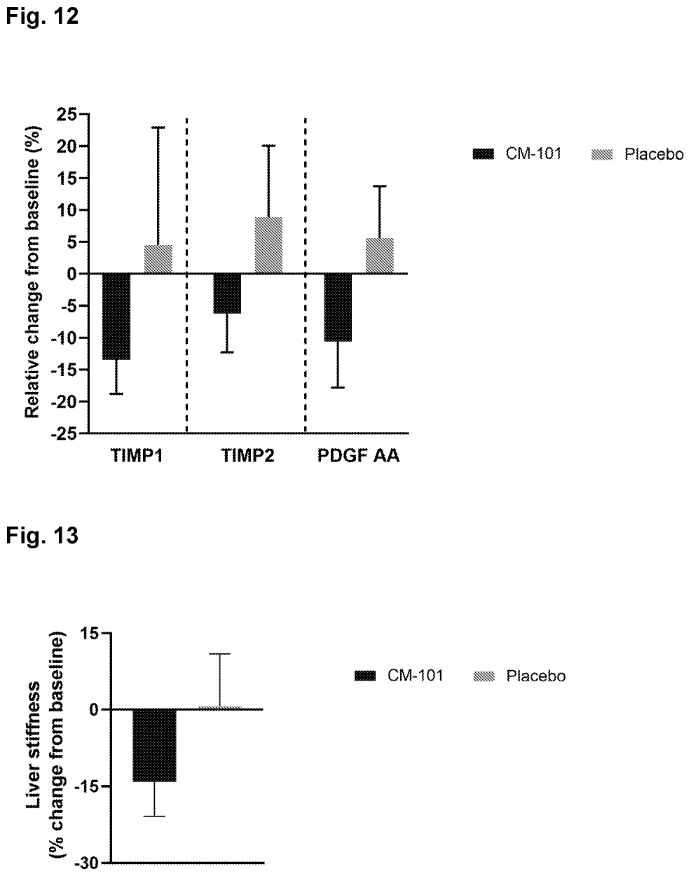

As shown in FIG. 11, CM-101 treatment resulted in reduction of Pro-C3 (−4.5%), Pro-C4 (−11.8%) and C3M (−4.7%) compared to no change or slight elevation in the placebo treated group. Similarly, as shown in FIG. 12, TIMP-1, TIMP-2 and PDGF AA were reduced by 10.5%, 4.4% and 10.6% respectively, while placebo group maintained a similar pattern of slight elevation. As shown in FIG. 13, FibroScan data collaborates with the effect on biomarkers with a mean reduction of 14.1% in liver stiffness.

1.-123. (canceled)

124. A method of treating a fibrotic inflammatory disease in a human subject, comprising administering an effective amount of a humanized anti-CCL24 monoclonal antibody or a pharmaceutical composition thereof, wherein the anti-CCL24 monoclonal antibody comprises a heavy chain having at least 95% sequence identity to SEQ ID NO: 11 and a light chain having at least 95% sequence identity to SEQ ID NO: 12; wherein said anti-CCL24 monoclonal antibody is administered at a dose of about 2.5 mg/kg, about 5 mg/kg, about 10 mg/kg, or about 20 mg/kg, and wherein the fibrotic inflammatory disease is non-alcoholic fatty liver disease (NAFLD), nonalcoholic steatohepatitis (NASH), cholestatic liver diseases, primary sclerosing cholangitis (PSC), or systemic sclerosis (SSC).

125. The method of claim 124, wherein the anti-CCL24 monoclonal antibody comprises a heavy chain according to SEQ ID NO: 11 and a light chain according to SEQ ID NO: 12.

126. The method of claim 124, wherein the anti-CCL24 monoclonal antibody is administered subcutaneously, or intravenously.

127. The method of claim 124, wherein the anti-CCL24 monoclonal antibody is administered once every one to four weeks, or once every two weeks, or once every three weeks, or once every four weeks.

128. The method of claim 124, wherein the anti-CCL24 monoclonal antibody is administered for at least four weeks, or for at least 15 weeks, or for at least 24 weeks.

129. The method of claim 124, wherein administration of the anti-CCL24 monoclonal antibody increases total sera level of CCL24 at day 4 post administration to at least 150% of baseline total sera level of CCL24 before administering the anti-CCL24 monoclonal antibody.

130. The method of claim 124, wherein said method further comprises a step of evaluating the level of one or more circulating fibrosis markers to determine improvement in the hepatic condition of the subject.

131. The method of claim 130, wherein the one or more circulating fibrosis markers are selected from the group consisting of plasma alanine aminotransferase (ALT), alkaline phosphatase (ALP), aspartate aminotransferase (AST), PRO-C3, PRO-C4, PRO-05, C3M, cytokeratin 18 (CK18), amino-terminal propeptide of type III procollagen (PIIINP), hyaluronic acid (HA), Tissue Inhibitor of Metalloproteinases 1 (TIMP-1), platelet derived growth factor (PDGF), Tissue Inhibitor of Metalloproteinases 2 (TIMP-2), γ-glutamyltransferase (GGT), bilirubin, C-reactive protein (CRP), erythrocyte sedimentation rate (ESR), and procalcitonin (PCT).

132. A pharmaceutical composition, comprising about 1 to about 500 mg/mL, or about 100 to about 250 mg/mL, or about 175 mg/mL of an anti-CCL24 monoclonal antibody comprising a heavy chain according to SEQ ID NO: 11 and a light chain according to SEQ ID NO: 12.

133. The pharmaceutical composition of claim 132, comprising a histidine buffer, optionally at about 10 mM.

134. The pharmaceutical composition of claim 132, comprising a sodium or potassium phosphate buffer, optionally at about 10 mM.

135. The pharmaceutical composition of claim 132, further comprising arginine, optionally at between about 30 and about 200 mM, or optionally at about 150 mM.

136. The pharmaceutical composition of claim 132, further comprising glutamic acid, optionally at between about 30 and about 200 mM, or optionally at about 150 mM.

137. The pharmaceutical composition of claim 132, further comprising sorbitol, optionally at about 4%.

138. The pharmaceutical composition of claim 132, further comprising glycine, optionally at about 10 mM to about 100 mM, or optionally about 40 mM.

139. The pharmaceutical composition of claim 132, further comprising polysorbate, optionally polysorbate-80, optionally at about 0.02% or about 0.01%.

140. The pharmaceutical composition of claim 132, wherein the pharmaceutical composition has a pH of 5 to 8, or 6 to 7, optionally 6.1 or 6.3.

141. The pharmaceutical composition of claim 132, wherein the pharmaceutical composition is supplied in a unit dose having a fill volume of about 0.2 to about 2 mL, or about 5 to about 20 mL.

142. The pharmaceutical composition of claim 132, comprising about 5 to about 20 mg/mL, or about 10 mg/mL of the anti-CCL24 monoclonal antibody.

143. A kit comprising the pharmaceutical composition of claim 132 and instructions for use.
